(19) United States
(12) Patent Application Publication
(10) Pub. No.: US 2026/0097045 A1
(43) Pub. Date: Apr. 9, 2026

(54) ANTIMICROBIAL COMBINATIONS

(71) Applicant: Helperby Therapeutics Limited, London Greater London (GB)

(72) Inventors: Robert J H Hammond, St Andrews Fife (GB); Anthony Coates, London (GB); Yanmin Hu, London (GB)

(73) Assignee: Helperby Therapeutics Limited, London Greater London (GB)

(21) Appl. No.: 19/113,206

(22) PCT Filed: Sep. 20, 2023
(86) PCT No.: PCT/GB23/52428

§ 371 Date: Mar. 19, 2025

(30) Foreign Application Priority Data
Sep. 20, 2022 (GB) 2213753.3

(51) Int. Cl. A61K 31/546; A61K 31/407; A61K 31/65; A61K 31/665; A61K 31/7072; A61K 38/12; A61P 31/04

(52) U.S. Cl. CPC A61K 31/546; A61K 31/407; A61K 31/65; A61K 31/665; A61K 31/7072; A61K 38/12; A61P 31/04

(57) ABSTRACT

The present invention provides an antimicrobial combination comprising three different antimicrobial agents The first antimicrobial agent is selected from ceftazidime, polymyxin E, polymyxin B, and pharmaceutically acceptable derivatives thereof; the second antimicrobial agent is selected from zidovudine, doxycycline, fosfomycin and pharmaceutically acceptable derivatives thereof; and the third antimicrobial agent is selected from levofloxacin, doxycycline, fosfomycin, meropenem, rifampicin, gentamicin, polymyxin B/E, and pharmaceutically acceptable derivatives thereof; wherein the combination includes at least one of levofloxacin, doxycycline, rifampicin, fosfomycin, or a pharmaceutically acceptable derivative thereof; provided the combination is not (1) polymyxin E/B, zidovudine and rifampicin or (2) ceftazidime, zidovudine and fosfomycin. Also provided is an antimicrobial combination comprising three antimicrobial agents, wherein the first antimicrobial agent is ceftazidime or a pharmaceutically acceptable derivative thereof; the second antimicrobial agent is zidovudine or a pharmaceutically acceptable derivative thereof; and the third antimicrobial agent is polymyxin E or a pharmaceutically acceptable derivative thereof.

### FIELD OF THE INVENTION

The present invention relates to a synergistic combination of three or four antimicrobial agents. The first, second and third agents (and fourth when present) are selected from respective groups as defined herein, and the combination is synergistic against gram-negative and/or gram-positive bacteria meaning that it is suitable for use in the treatment of microbial infections caused by said bacteria. In particular, the present invention relates to the use of such combinations to kill multiplying (i.e. log phase) microorganisms associated with bacterial infections, e.g. Gram-negative bacterial infections.

### BACKGROUND

Before the introduction of antibiotics, patients suffering from acute microbial infections (e.g. tuberculosis or pneumonia) had a low chance of survival. For example, mortality from tuberculosis was around 50%. The introduction of antimicrobial agents in the 1940s and 1950s rapidly changed this picture, and now there are approximately 100 antibiotics being used to treat different bacterial infections. This has enabled the creation of modern medicine because bacterial infections can be prevented and treated effectively in millions of patients with e.g. cancer, organ transplantation, kidney dialysis, immunosuppression and surgery.

Bacteria have, however, responded to the widespread use of antibiotics by progressively gaining resistance. Now, every country in the world has antibiotic-resistant bacteria and this resistance increases annually, thereby decreasing the effectiveness of all antibiotics. By 2040-2050, antimicrobial resistance infection deaths are predicted to exceed 10 million per year (The Review on Antimicrobial Resistance, Chaired by Jim O'Neill, May 2016).

The increase in antimicrobial resistance is expected to occur in poorer countries earlier than 2040-2050, and is already a real, practical, medical issue in Europe and the United States of America. Indeed, more than 70% of bacteria that give rise to hospital acquired infections in the USA resist at least one of the main antimicrobial agents that are typically used to fight infection (Nature Reviews, Drug Discovery, 1, 895-910 (2002)). The World Health Organization has therefore classified antimicrobial resistance as a “serious threat [that] is no longer a prediction for the future, it is happening right now in every region of the world and has the potential to affect anyone, of any age, in any country” (“Antimicrobial resistance: global report on surveillance”, The World Health Organization, April 2014). If not addressed, life expectancy could revert to pre-antibiotic levels, namely about 20 years less than todays.

A solution to the growing problem of resistant bacteria is therefore desperately needed. In essence, the medical field needs to replace the circa. 100 antibiotics with products effective against both antimicrobial resistant infections whilst avoiding the development of future antimicrobial resistance.

Current efforts to solve this problem largely concentrate on the development of new chemical entities or NCEs. Each NCE requires more than 10 years' development and costs in excess of $600 million to complete the necessary safety and clinical testing. Substantial numbers fail and hence about $3.8 billion is typically required to deliver one NCE antibiotic. Ironically, antimicrobial resistance also develops faster as the antibiotic use increases meaning that all NCEs have a limited useful life, often less than 10 years. Replacing the currently used antibiotics with NCEs would thus require approximately $3,800 billion within a 10 year period and if successful, these products would then require continued plans to replace them within their 10 year life as antimicrobial resistance develops to each one. This is clearly unsustainable, even for higher income countries.

The Applicant has identified a solution to this significant and worldwide problem. Specifically, combinations of three or more known antibiotics, surprisingly identified to have synergy against gram-negative and/or gram-positive bacteria. Such combinations have been found to kill antimicrobial-resistant bacteria, also termed drug-resistant bacteria, and avoid the development of antimicrobial resistance. The three or more known antibiotics are defined in the appended claims and described herein.

The synergy demonstrated means that the combinations have a greater biological activity than the expected additive effect of each agent at the stated dosage level. This means that lower amounts, such as 5 to 20×less antibiotic, are more effect in the combination therapy than any of the single antibiotics.

Furthermore, by combining already approved drugs (CADs), the time to approval and the cost involved is much less than NCEs. The time is, for example, about 5 years, and no more than $50M is needed to produce one new product. Since far fewer fail in development, the accumulated success/fail costs are about $130M for one CAD compared to $3.8 billion for each NCE. To replace the circa. 100 single antibiotics which are used today, the number of combinations required can be reduced by virtue of CADs having activity against a broad range of different infective species and can rejuvenate a single, unusable antibiotic due to antimicrobial resistance within a CAD with much higher activity against these antimicrobial resistant strains and avoid future antimicrobial resistance developing. As a proportion of GDP, the CAD technology moves from 4% of high income countries for NCEs to about 0.005% GDP, allowing all high, middle and even low income countries to contribute as well as benefit.

The current, global focus of exclusively repeated NCE programs with their much higher cost/time/failure potential and then short usable life, predicts effective antibiotics being exhausted within two decades, and antimicrobial resistance deaths returning us 200 years backwards. Investing even 0.1% of the NCE development costs in the CAD approach provides a practical, affordable and longer lasting solution which offers a realistic option to continue to provide useable antibiotic products—globally effective and affordable—giving hope of “Affordable Antibiotics Forever and For All”.

Over the last decade, the Applicant, a small UK company and one not-for profit organisation (GARDP) are actively developing the CAD-antibiotic combinations of existing drugs. In the present application, the Applicants have developed a new approach for replacing all antibiotics by discovering synergistic combinations of three antimicrobial agents. The combinations are active against at least Extended Spectrum Beta-Lactamase (ESBL), carbapenem-producing (CPE) and carbapenem-resistant (CRE) gram-negative bacteria. Even more surprisingly, the Applicant has revealed that the concentration of each antimicrobial agent of the combination is active at a very low concentration, for example, as low as 1/16^{th} of the agent's MICₘₒₙₒ. Thus, it may advantageously be possible to reduce any potential burden of toxicity by using lower amounts of each antimicrobial agent in the combination than e.g. for a monotherapy.

WO2015/114340 describes the use of zidovudine in combination with a polymyxin selected from colistin or polymyxin B, an anti-tuberculosis antibiotic selected from rifampicin, rifapentine or rifabutin and optionally piperine, for treating a microbial infection. WO2018/011562 describes a combination comprising zidovudine and a carbapenem, optionally with a polymyxin selected from polymyxin B and polymyxin E. The present invention does not therefore encompass these previously identified combinations of the Applicant.

Synergy is not predictable or expected when two or more actives are used in combination. Synergy in the context of antimicrobial drugs is measured in a number of ways that conform to the generally accepted opinion that “synergy is an effect greater than additive”. One of the ways to assess whether synergy has been observed is to use the “chequerboard” technique. This is a well-accepted method that leads to the generation of a value called the fractional inhibitory concentration index (FICI). Orhan et al., J. Clin. Microbiol. 2005, 43 (1): 140 describes the chequerboard method and analysis in the paragraph bridging pages 140-141.

FICI or fractional inhibitory concentration index is the sum of the FICs of each antimicrobial when used in combination. The FIC or fractional inhibitory concentration of an antimicrobial in a combination, is the MIC of the antimicrobial in the combination divided by the MIC of the same antimicrobial when used alone. Minimum inhibitory concentrations are defined in the art as the lowest concentration of an antimicrobial that will inhibit the visible growth of a microorganism after overnight incubation.

The combinations are active against resistant bacteria (see the Examples herein). The individual antimicrobial agents in these combinations are notably active in many cases at concentrations significantly below the MIC when used alone. There does not, however, appear to be a method in the art for defining synergy of a 3-mer (a combination including three antimicrobial agents) or a 4-mer (a combination including four antimicrobial agents) expressed as Fractional Inhibitory Concentration. There has in fact been very little previous work in this field. Hence, the present inventors devised such a method. This method is applicable to any combination with more than two antimicrobial agents; provided that there are only two antimicrobial agents which are varied in their concentration.

In the method for a 3-mer combination, one antibiotic was “fixed” as part of a backbone whilst the two other antibiotics were varied over a doubling concentration scale beginning at the MIC (×1) for the effective dose in monotherapy against the organism being tested. The ΣFIC was then calculated as shown below. In the method for a 4-mer combination, two antibiotics were “fixed” as part of a backbone whilst the two other antibiotics were varied over a doubling concentration scale beginning at the MIC (×1) for the effective dose in monotherapy against the organism being tested.

The ΣFIC was divided by “0.5/n” where n=number of antimicrobials in the combination to provide FIC. The inventors opted for this expression because it more closely aligns with the 2-mer synergy levels. The same FIC scale as used for 2-mers was applied: synergy is observed when FIC is <0.5. An “Additive” effect is observed when FIC is 0.5 to <1. Indifference is observed when FIC is 1 to <2. Antagonism is observed when FIC is 2 to 4.

Synergy can be expressed as “ΣFIC≤0.25×n”.

| | MIC | | | |
|---|---|---|---|---|
| | Monotherapy | MIC combination | | |
| | (X) | therapy (Y) | Ratio Y:X | |
| Drug A | 4 | 1 | 0.25 | |
| Drug B | 1 | 0.25 | 0.25 | |
| Drug C | 2 | 0.25 | 0.125 | |
| | | TOTAL | 0.625 | ΣFIC |
| | | (TOTAL/0.5)/[number | 0.417 | FIC |
| | | of drugs] | | |

The above method was used in the Examples herein.

### SUMMARY OF THE INVENTION

In one aspect the present invention provides an antimicrobial combination comprising three antimicrobial agents, wherein (i) the first antimicrobial agent is selected from ceftazidime, polymyxin E, polymyxin B, and pharmaceutically acceptable derivatives thereof, (ii) the second antimicrobial agent is selected from zidovudine, doxycycline, fosfomycin and pharmaceutically acceptable derivatives thereof, and (iii) the third antimicrobial agent is selected from levofloxacin, doxycycline, fosfomycin, meropenem, rifampicin, gentamicin, polymyxin B/E, and pharmaceutically acceptable derivatives thereof. The first, second and third antimicrobial agents in the combination are different from one another and the combination includes at least one of levofloxacin, doxycycline, rifampicin, fosfomycin, or a pharmaceutically acceptable derivative thereof, provided the combination is not (1) polymyxin E/B, zidovudine and rifampicin, or (2) ceftazidime, zidovudine and fosfomycin. In further embodiments of this aspect, the combination includes a fourth antimicrobial agent which is a carbapenem or a pharmaceutically acceptable derivative thereof, preferably meropenem or a pharmaceutically acceptable derivative thereof.

In another aspect the present invention provides an antimicrobial combination comprising three antimicrobial agents. The first antimicrobial agent is ceftazidime or a pharmaceutically acceptable derivative thereof, the second antimicrobial agent is zidovudine or a pharmaceutically acceptable derivative thereof, and the third antimicrobial agent is polymyxin E or a pharmaceutically acceptable derivative thereof. In further embodiments of this aspect, the combination includes a fourth antimicrobial agent which is a carbapenem or a pharmaceutically acceptable derivative thereof, preferably meropenem or a pharmaceutically acceptable derivative thereof.

In another aspect the present invention provides an antimicrobial combination comprising three antimicrobial agents wherein the first antimicrobial agent is ceftazidime or a pharmaceutically acceptable derivative thereof; the second antimicrobial agent is zidovudine, levofloxacin, or a pharmaceutically acceptable derivative thereof; and the third antimicrobial agent is meropenem or a pharmaceutically acceptable derivative thereof.

In another aspect the present invention provides the combinations for use in treating an infection caused by gram-negative or gram-positive bacteria.

In another aspect the present invention provides a pharmaceutical composition comprising the combination defined herein and a pharmaceutically acceptable adjuvant, diluent and carrier. The pharmaceutical composition is preferably for use in the treatment of an infection caused by gram-negative or gram-positive bacteria.

In another aspect the present invention provides a product comprising an antimicrobial combination of three antimicrobial agents, wherein: the first antimicrobial agent is selected from ceftazidime, polymyxin E, polymyxin B, and pharmaceutically acceptable derivatives thereof; the second antimicrobial agent is selected from zidovudine, doxycycline, fosfomycin, and pharmaceutically acceptable derivatives thereof; and the third antimicrobial agent is selected from levofloxacin, doxycycline, meropenem, rifampicin, fosfomycin, gentamicin, polymyxin B, polymyxin E, and pharmaceutically acceptable derivatives thereof; wherein the first, second and third antimicrobial agents in the combination are different from one another; wherein the combination includes at least levofloxacin, doxycycline, rifampicin, fosfomycin or a pharmaceutically acceptable derivative thereof; provided the combination is not (1) polymyxin E/B, zidovudine and rifampicin or (2) ceftazidime, zidovudine and fosfomycin; as a combined preparation for simultaneous, separate or sequential use in treating an infection caused by gram-negative or gram-positive bacteria. In further embodiments of this aspect, the combination includes a fourth antimicrobial agent which is a carbapenem or a pharmaceutically acceptable derivative thereof, preferably meropenem or a pharmaceutically acceptable derivative thereof.

In another aspect the present invention provides a product comprising an antimicrobial combination of three antimicrobial agents, wherein the first antimicrobial agent is ceftazidime or a pharmaceutically acceptable derivative thereof; the second antimicrobial agent is zidovudine or a pharmaceutically acceptable derivative thereof; and the third antimicrobial agent is polymyxin E or a pharmaceutically acceptable derivative thereof, as a combined preparation for simultaneous, separate or sequential use in treating an infection caused by gram-negative or gram-positive bacteria. In further embodiments of this aspect, the combination includes a fourth antimicrobial agent which is a carbapenem or a pharmaceutically acceptable derivative thereof, preferably meropenem or a pharmaceutically acceptable derivative thereof.

In another aspect the present invention provides a product comprising an antimicrobial combination of three antimicrobial agents, wherein the first antimicrobial agent is ceftazidime or a pharmaceutically acceptable derivative thereof; the second antimicrobial agent is zidovudine, levofloxacin, or a pharmaceutically acceptable derivative thereof; and the third antimicrobial agent is meropenem or a pharmaceutically acceptable derivative thereof, as a combined preparation for simultaneous, separate or sequential use in treating an infection caused by gram-negative or gram-positive bacteria.

In another aspect the present invention provides the use of a first antimicrobial agent in combination with at least a second and third antimicrobial agents in the manufacture of a medicament for synergistically treating a gram-negative or gram-positive bacterial infection. The first antimicrobial agent is selected from ceftazidime, polymyxin E, polymyxin B, and pharmaceutically acceptable derivatives thereof. The second antimicrobial agent is selected from zidovudine, doxycycline, fosfomycin, and pharmaceutically acceptable derivatives thereof. The third antimicrobial agent is selected from levofloxacin, doxycycline, meropenem, rifampicin, gentamicin, polymyxin B, polymyxin E, and pharmaceutically acceptable derivatives thereof. The combination includes at least levofloxacin, rifampicin, doxycycline, fosfomycin or a pharmaceutically acceptable derivative thereof, the first, second and third antimicrobial agents are different from one another, provided the combination is not polymyxin E/B, zidovudine and rifampicin. In a further embodiment of this aspect, the combination includes a fourth antimicrobial agent which is a carbapenem or a pharmaceutically acceptable derivative thereof, preferably meropenem or a pharmaceutically acceptable derivative thereof.

In another aspect the present invention provides the use of a second antimicrobial agent in combination with at least a first and third antimicrobial agents in the manufacture of a medicament for synergistically treating a gram-negative or gram-positive bacterial infection. The first antimicrobial agent is selected from ceftazidime, polymyxin E, polymyxin B, and pharmaceutically acceptable derivatives thereof. The second antimicrobial agent is selected from zidovudine, doxycycline, fosfomycin, and pharmaceutically acceptable derivatives thereof. The third antimicrobial agent is selected from levofloxacin, doxycycline, meropenem, rifampicin, gentamicin, polymyxin B, polymyxin E, and pharmaceutically acceptable derivatives thereof. The combination includes at least levofloxacin, doxycycline, rifampicin, fosfomycin or a pharmaceutically acceptable derivative thereof, the first, second and third antimicrobial agents are different from one another, provided the combination is not polymyxin E/B, zidovudine and rifampicin. In a further embodiment of this aspect, the combination includes a fourth antimicrobial agent which is a carbapenem or a pharmaceutically acceptable derivative thereof, preferably meropenem or a pharmaceutically acceptable derivative thereof.

In another aspect the present invention provides the use of a third antimicrobial agent in combination with at least a first and second antimicrobial agents in the manufacture of a medicament for synergistically treating a gram-negative or gram-positive bacterial infection. The first antimicrobial agent is selected from ceftazidime, polymyxin E, polymyxin B, and pharmaceutically acceptable derivatives thereof. The second antimicrobial agent is selected from zidovudine, doxycycline, fosfomycin, and pharmaceutically acceptable derivatives thereof. The third antimicrobial agent is selected from levofloxacin, doxycycline, meropenem, rifampicin, gentamicin, polymyxin B, polymyxin E, and pharmaceutically acceptable derivatives thereof. The combination includes at least levofloxacin, doxycycline, rifampicin, fosfomycin or a pharmaceutically acceptable derivative thereof, the first, second and third antimicrobial agents are different from one another, provided the combination is not polymyxin E/B, zidovudine and rifampicin. In a further embodiment of this aspect, the combination includes a fourth antimicrobial agent which is a carbapenem or a pharmaceutically acceptable derivative thereof, preferably meropenem or a pharmaceutically acceptable derivative thereof.

In another aspect the present invention provides the use of ceftazidime or a pharmaceutically acceptable derivative thereof, in combination with zidovudine or a pharmaceutically acceptable derivative thereof and polymyxin E or a pharmaceutically acceptable derivative thereof, in the manufacture of a medicament for synergistically treating a gram-negative or gram-positive bacterial infection. In a further embodiment of this aspect, the combination also includes a carbapenem or a pharmaceutically acceptable derivative thereof, preferably meropenem or a pharmaceutically acceptable derivative thereof.

In another aspect the present invention provides the use of zidovudine or a pharmaceutically acceptable derivative thereof, in combination with ceftazidime or a pharmaceutically acceptable derivative thereof and polymyxin E or a pharmaceutically acceptable derivative thereof, in the manufacture of a medicament for synergistically treating a gram-negative or gram-positive bacterial infection. In a further embodiment of this aspect, the combination also includes a carbapenem or a pharmaceutically acceptable derivative thereof, preferably meropenem or a pharmaceutically acceptable derivative thereof.

In another aspect the present invention provides the use of polymyxin E or a pharmaceutically acceptable derivative thereof, in combination with zidovudine or a pharmaceutically acceptable derivative thereof and ceftazidime or a pharmaceutically acceptable derivative thereof, in the manufacture of a medicament for synergistically treating a gram-negative or gram-positive bacterial infection. In a further embodiment of this aspect, the combination also includes a carbapenem or a pharmaceutically acceptable derivative thereof, preferably meropenem or a pharmaceutically acceptable derivative thereof.

In another aspect the present invention provides the use of ceftazidime or a pharmaceutically acceptable derivative thereof, in combination with zidovudine, levofloxacin, or a pharmaceutically acceptable derivative thereof, and meropenem or a pharmaceutically acceptable derivative thereof, in the manufacture of a medicament for synergistically treating a gram-negative or gram-positive bacterial infection.

In another aspect the present invention provides the use of zidovudine, levofloxacin, or a pharmaceutically acceptable derivative thereof, in combination with ceftazidime or a pharmaceutically acceptable derivative thereof, and meropenem or a pharmaceutically acceptable derivative thereof, in the manufacture of a medicament for synergistically treating a gram-negative or gram-positive bacterial infection.

In another aspect the present invention provides the use of meropenem or a pharmaceutically acceptable derivative thereof, in combination with zidovudine, levofloxacin, or a pharmaceutically acceptable derivative thereof, and ceftazidime or a pharmaceutically acceptable derivative thereof, in the manufacture of a medicament for synergistically treating a gram-negative or gram-positive bacterial infection.

In another aspect the present invention provides a method of treating a gram-negative or gram-positive bacterial infection, wherein the method comprises administering a pharmaceutically effective amount of a combination comprising three antimicrobial agents, wherein the first antimicrobial agent is selected from ceftazidime, polymyxin E, polymyxin B, and pharmaceutically acceptable derivatives thereof; the second antimicrobial agent is selected from zidovudine, doxycycline, fosfomycin, and pharmaceutically acceptable derivatives thereof; and the third antimicrobial agent is selected from levofloxacin, doxycycline, meropenem, rifampicin, gentamicin, polymyxin B, polymyxin E, and pharmaceutically acceptable derivatives thereof; wherein the first, second and third antimicrobial agents in the combination are different from one another; wherein the combination includes at least levofloxacin, doxycycline, rifampicin, fosfomycin or a pharmaceutically acceptable derivative thereof; provided the combination is not polymyxin E/B, zidovudine and rifampicin. In a further embodiment of this aspect, the combination also includes a carbapenem or a pharmaceutically acceptable derivative thereof, preferably meropenem or a pharmaceutically acceptable derivative thereof.

In another aspect the present invention provides a method of treating a gram-negative or gram-positive bacterial infection, wherein the method comprises administering a pharmaceutically effective amount of a combination comprising three antimicrobial agents, wherein the first antimicrobial agent is ceftazidime or a pharmaceutically acceptable derivative thereof; the second antimicrobial agent is zidovudine, levofloxacin, or a pharmaceutically acceptable derivative thereof; and the third antimicrobial agent is meropenem or a pharmaceutically acceptable derivative thereof.

These aspects and embodiments thereof are set out in the appended independent and dependent claims. It will be appreciated that features of the dependent claims may be combined with each other and with features of the independent claims in combinations other than those explicitly set out in the claims. Furthermore, the present disclosure is not restricted to the specific embodiments set out below, but includes and contemplates any combinations of features presented herein.

The foregoing and other aspects, embodiments, features and advantages of the present disclosure will be apparent from the following detailed description. In this regard, particular sections of the description are not to be read in isolation from other sections.

### DETAILED DESCRIPTION

While various exemplary embodiments are described or suggested herein, other exemplary embodiments utilizing a variety of methods and materials similar or equivalent to those described or suggested herein are encompassed by the general inventive concepts. Those features or embodiments which are implemented conventionally may not be discussed or described in detail in the interests of brevity. It will thus be appreciated that features of apparatus, products or processes described herein which are not described in detail may be implemented in accordance with any conventional techniques for implementing such features in the respective context.

As used herein, the expressions “combination of” and “in combination with” cover separate, sequential and simultaneous administration of the agents. Unless specified to the contrary, the expressions are also intended to exclude any additional actives, e.g. “a synergistic combination comprising three antimicrobial agents” means that the defined antimicrobial agents are administered separately, sequentially or simultaneously but that no other actives, i.e. antimicrobial agents, are administered.

When the agents are administered sequentially, either the first, second or third antimicrobial agent may be administered first. When administration is simultaneous, the agents may be administered either in the same or a different pharmaceutical composition. In preferred embodiments, the agents are administered sequentially or simultaneously.

The combinations of the present invention may be used to treat gram-positive or gram-negative bacterial infections. In particular, they may be used to kill multiplying and/or clinically latent bacteria associated with such infections, preferably multiplying bacteria associated with such infections, e.g. multiplying bacteria associated with Gram-negative bacterial infections. References herein to the treatment of a bacterial infection therefore include killing multiplying and/or clinically latent microorganisms associated with such infections.

As used herein, “kill” means a loss of viability as assessed by a lack of metabolic activity.

As used herein, “clinically latent bacteria” means bacteria that is metabolically active but has a growth rate that is below the threshold of infectious disease expression. The threshold of infectious disease expression refers to the growth rate threshold below which symptoms of infectious disease in a host are absent.

The metabolic activity of clinically latent bacteria can be determined by several methods known to those skilled in the art; for example, by measuring mRNA levels in the bacteria or by determining their rate of uridine uptake. In this respect, clinically latent bacteria, when compared to bacteria under logarithmic growth conditions (in vitro or in vivo), possess reduced but still significant levels of:
(1) mRNA (e.g. from 0.0001 to 50%, such as from 1 to 30, 5 to 25 or 10 to 20%, of the level of mRNA); and/or
(II) uridine (e.g. [3H]uridine) uptake (e.g. from 0.0005 to 50%, such as from 1 to 40, 15 to 35 or 20 to 30% of the level of [3H]uridine uptake).

Clinically latent bacteria typically possess a number of identifiable characteristics. For example, they may be viable but non-culturable; i.e. they cannot typically be detected by standard culture techniques, but are detectable and quantifiable by techniques such as broth dilution counting, microscopy, or molecular techniques such as polymerase chain reaction. In addition, clinically latent bacteria are phenotypically tolerant, and as such are sensitive (in log phase) to the biostatic effects of conventional antimicrobial agents (i.e. bacteria for which the minimum inhibitory concentration (MIC) of a conventional antimicrobial is substantially unchanged); but possess drastically decreased susceptibility to drug-induced killing (e.g. bacteria for which, with any given conventional antimicrobial agent, the ratio of minimum microbiocidal concentration (e.g. minimum bactericidal concentration, MBC) to MIC is 10 or more).

In various embodiments of the invention, one or more of the aforementioned combinations is used to treat a bacterial infection, in particular the combinations may be used to kill multiplying and/or clinically latent bacteria associated with the bacterial infection. As used herein, the term “bacteria” (and derivatives thereof, such as “bacterial infection”) includes, but is not limited to, references to organisms (or infections due to organisms) of the following classes and specific types:

Gram-positive cocci, such as Staphylococci (e.g. Staph. aureus, Staph. epidermidis, Staph. saprophyticus, Staph. auricularis, Staph. capitis capitis, Staph. c. ureolyticus, Staph. caprae, Staph. cohnii cohnii, Staph. c. urealyticus, Staph. equorum, Staph. gallinarum, Staph. haemolyticus, Staph. hominis hominis, Staph. h. novobiosepticius, Staph. hyicus, Staph. intermedius, Staph. lugdunensis, Staph. pasteuri, Staph. saccharolyticus, Staph. schleiferi schleiferi, Staph. s. coagulans, Staph. sciuri, Staph. simulans, Staph. warneri and Staph. xylosus); Streptococci (e.g. beta-haemolytic, pyogenic streptococci (such as Strept. agalactiae, Strept. canis, Strept. dysgalactiae dysgalactiae, Strept. dysgalactiae equisimilis, Strept. equi equi, Strept. equi zooepidemicus, Strept. iniae, Strept. porcinus and Strept. pyogenes), microaerophilic, pyogenic streptococci (Streptococcus “milleri”, such as Strept. anginosus, Strept. constellatus constellatus, Strept. constellatus pharyngidis and Strept. intermedius), oral streptococci of the “mitis” (alpha-haemolytic-Streptococcus “viridans”, such as Strept. mitis, Strept. oralis, Strept. sanguinis, Strept. cristatus, Strept. gordonii and Strept. parasanguinis), “salivarius” (non-haemolytic, such as Strept. salivarius and Strept. vestibularis) and “mutans” (tooth-surface streptococci, such as Strept. criceti, Strept. mutans, Strept. ratti and Strept. sobrinus) groups, Strept. acidominimus, Strept. bovis, Strept. faecalis, Strept. equinus, Strept. pneumoniae and Strept. suis, or Streptococci alternatively classified as Group A, B, C, D, E, G, L, P, U or V Streptococcus); Enterococci (e.g. Enterococcus avium, Enterococcus casseliflavus, Enterococcus cecorum, Enterococcus dispar, Enterococcus durans, Enterococcus faecalis, Enterococcus faecium, Enterococcus flavescens, Enterococcus gallinarum, Enterococcus hirae, Enterococcus malodoratus, Enterococcus mundtii, Enterococcus pseudoavium, Enterococcus raffinosus and Enterococcus solitarius); Bacillaceae, such as Bacillus anthracis, Bacillus subtilis, Bacillus thuringiensis, Bacillus stearothermophilus and Bacillus cereus;

Gram-negative cocci, such as Neisseria gonorrhoeae, Neisseria meningitidis, Neisseria cinerea, Neisseria elongata, Neisseria flavescens, Neisseria lactamica, Neisseria mucosa, Neisseria sicca, Neisseria subflava and Neisseria weaveri; Enterobacteriaceae, such as Escherichia coli, Enterobacter (e.g. Enterobacter aerogenes, Enterobacter agglomerans and Enterobacter cloacae), Citrobacter (such as Citrob. freundii and Citrob. divernis), Hafnia (e.g. Hafnia alvei), Erwinia (e.g. Erwinia persicinus), Morganella (e.g. Morganella morganii), Salmonella (Salmonella enterica and Salmonella typhi), Shigella (e.g. Shigella dysenteriae, Shigella flexneri, Shigella boydii and Shigella sonnei), Klebsiella (e.g. Klebs. pneumoniae, Klebs. oxytoca, Klebs. ornitholytica, Klebs. planticola, Klebs. ozaenae, Klebs. terrigena, Klebs. granulomatis (Calymmatobacterium granulomatis) and Klebs. rhinoscleromatis), Proteus (e.g. Pr. mirabilis, Pr. rettgeri and Pr. vulgaris), Providencia (e.g. Providencia alcalifaciens, Providencia rettgeri and Providencia stuartii), Serratia (e.g. Serratia marcescens and Serratia liquifaciens), and Yersinia (e.g. Yersinia enterocolitica, Yersinia pestis and Yersinia pseudotuberculosis); Helicobacter (e.g. Helicobacter pylori, Helicobacter cinaedi and Helicobacter fennelliae); Acinetobacter (e.g. A. baumanii, A. calcoaceticus, A. haemolyticus, A. johnsonii, A. junii, A. lwoffi and A. radioresistens); Pseudomonas (e.g. Ps. aeruginosa, Ps. maltophilia (Stenotrophomonas maltophilia), Ps. alcaligenes, Ps. chlororaphis, Ps. fluorescens, Ps. luteola. Ps. mendocina, Ps. monteilii, Ps. oryzihabitans, Ps. pertocinogena, Ps. pseudalcaligenes, Ps. putida and Ps. stutzen); Bacteroides fragilis; Peptococcus (e.g. Peptococcus niger); Peptostreptococcus; Clostridium (e.g. C. perfringens, C. difficile, C. botulinum, C. tetani, C. absonum, C. argentinense, C. baratii, C. bifermentans, C. beijerinckii, C. butyricum, C. cadaveris, C. carnis, C. celatum, C. clostridioforme, C. cochlearium, C. cocleatum, C. fallax, C. ghonii, C. glycolicum, C. haemolyticum, C. hastiforme, C. histolyticum, C. indolis, C. innocuum, C. irregulare, C. leptum, C. limosum, C. malenominatum, C. novyi, C. oroticum, C. paraputrificum, C. piliforme, C. putrefasciens, C. ramosum, C. septicum, C. sordelii, C. sphenoides, C. sporogenes, C. subterminale, C. symbiosum and C. tertium); Mycoplasma (e.g. M. pneumoniae, M. hominis, M. genitalium and M. urealyticum); Mycobacteria (e.g. Mycobacterium tuberculosis, Mycobacterium avium, Mycobacterium fortuitum, Mycobacterium marinum, Mycobacterium kansasii, Mycobacterium chelonae, Mycobacterium abscessus, Mycobacterium leprae, Mycobacterium smegmitis, Mycobacterium africanum, Mycobacterium alvei, Mycobacterium asiaticum, Mycobacterium aurum, Mycobacterium bohemicum, Mycobacterium bovis, Mycobacterium branderi, Mycobacterium brumae, Mycobacterium celatum, Mycobacterium chubense, Mycobacterium confluentis, Mycobacterium conspicuum, Mycobacterium cookii, Mycobacterium flavescens, Mycobacterium gadium, Mycobacterium gastri, Mycobacterium genavense, Mycobacterium gordonae, Mycobacterium goodii, Mycobacterium haemophilum, Mycobacterium hassicum, Mycobacterium intracellulare, Mycobacterium interjectum, Mycobacterium heidelberense, Mycobacterium lentiflavum, Mycobacterium malmoense, Mycobacterium microgenicum, Mycobacterium microti, Mycobacterium mucogenicum, Mycobacterium neoaurum, Mycobacterium nonchromogenicum, Mycobacterium peregrinum, Mycobacterium phlei, Mycobacterium scrofulaceum, Mycobacterium shimoidei, Mycobacterium simiae, Mycobacterium szulgai, Mycobacterium terrae, Mycobacterium thermoresistabile, Mycobacterium triplex, Mycobacterium triviale, Mycobacterium tusciae, Mycobacterium ulcerans, Mycobacterium vaccae, Mycobacterium wolinskyi and Mycobacterium xenopi); Haemophilus (e.g. Haemophilus influenzae, Haemophilus ducreyi, Haemophilus aegyptius, Haemophilus parainfluenzae, Haemophilus haemolyticus and Haemophilus parahaemolyticus); Actinobacillus (e.g. Actinobacillus actinomycetemcomitans, Actinobacillus equuli, Actinobacillus hominis, Actinobacillus lignieresii, Actinobacillus suis and Actinobacillus ureae); Actinomyces (e.g. Actinomyces israelii); Brucella (e.g. Brucella abortus, Brucella canis, Brucella melintensis and Brucella suis); Campylobacter (e.g. Campylobacter jejuni, Campylobacter coli, Campylobacter Iari and Campylobacter fetus); Listeria monocytogenes; Vibrio (e.g. Vibrio cholerae and Vibrio parahaemolyticus, Vibrio alginolyticus, Vibrio carchariae, Vibrio fluvialis, Vibrio furnissii, Vibrio hollisae, Vibrio metschnikovii, Vibrio mimicus and Vibrio vulnificus); Erysipelothrix rhusopathiae; Corynebacteriaceae (e.g. Corynebacterium diphtheriae, Corynebacterium jeikeum and Corynebacterium urealyticum); Spirochaetaceae, such as Borrelia (e.g. Borrelia recurrentis, Borrelia burgdorferi, Borrelia afzelii, Borrelia andersonii, Borrelia bissettii, Borrelia garinii, Borrelia japonica, Borrelia lusitaniae, Borrelia tanukii, Borrelia turdi, Borrelia valaisiana, Borrelia caucasica, Borrelia crocidurae, Borrelia duttoni, Borrelia graingeri, Borrelia hermsii, Borrelia hispanica, Borrelia latyschewii, Borrelia mazzottii, Borrelia parkeri, Borrelia persica, Borrelia turicatae and Borrelia venezuelensis) and Treponema (Treponema pallidum ssp. pallidum, Treponema pallidum ssp. endemicum, Treponema pallidum ssp. pertenue and Treponema carateum); Pasteurella (e.g. Pasteurella aerogenes, Pasteurella bettyae, Pasteurella canis, Pasteurella dagmatis, Pasteurella gallinarum, Pasteurella haemolytica, Pasteurella multocida multocida, Pasteurella multocida gallicida, Pasteurella multocida septica, Pasteurella pneumotropica and Pasteurella stomatis); Bordetella (e.g. Bordetella bronchiseptica, Bordetella hinzii, Bordetella holmseii, Bordetella parapertussis, Bordetella pertussis and Bordetella trematum); Nocardiaceae, such as Nocardia (e.g. Nocardia asteroides and Nocardia brasiliensis); Rickettsia (e.g. Ricksettsii or Coxiella burnetii); Legionella (e.g. Legionalla anisa, Legionalla birminghamensis, Legionalla bozemanii, Legionalla cincinnatiensis, Legionalla dumoffii, Legionalla feeleii, Legionalla gormanii, Legionalla hackeliae, Legionalla israelensis, Legionalla jordanis, Legionalla lansingensis, Legionalla Iongbeachae, Legionalla maceachernii, Legionalla micdadei, Legionalla oakridgensis, Legionalla pneumophila, Legionalla sainthelensi, Legionalla tucsonensis and Legionalla wadsworthii); Moraxella catarrhalis; Cyclospora cayetanensis; Entamoeba histolytica; Giardia lamblia; Trichomonas vaginalis; Toxoplasma gondii; Stenotrophomonas maltophilia; Burkholderia stenotrophomonas; Burkholderia cepacia; Burkholderia mallei and Burkholderia pseudomallei; Francisella tularensis; Gardnerella (e.g. Gardnerella vaginalis and Gardnerella mobiluncus); Streptobacillus moniliformis; Flavobacteriaceae, such as Capnocytophaga (e.g. Capnocytophaga canimorsus, Capnocytophaga cynodegmi, Capnocytophaga gingivalis, Capnocytophaga granulosa, Capnocytophaga haemolytica, Capnocytophaga ochracea and Capnocytophaga sputigena); Bartonella (Bartonella bacilliformis, Bartonella clarridgeiae, Bartonella elizabethae, Bartonella henselae, Bartonella quintana and Bartonella vinsonii arupensis); Leptospira (e.g. Leptospira biflexa, Leptospira borgpetersenii, Leptospira inadai, Leptospira interrogans, Leptospira kirschneri, Leptospira noguchii, Leptospira santarosai and Leptospira weilii); Spirillum (e.g. Spirillum minus); Baceteroides (e.g. Bacteroides caccae, Bacteroides capillosus, Bacteroides coagulans, Bacteroides distasonis, Bacteroides eggerthii, Bacteroides forsythus, Bacteroides fragilis, Bacteroides merdae, Bacteroides ovatus, Bacteroides putredinis, Bacteroides pyogenes, Bacteroides splanchinicus, Bacteroides stercoris, Bacteroides tectus, Bacteroides thetaiotaomicron, Bacteroides uniformis, Bacteroides ureolyticus and Bacteroides vulgatus); Prevotella (e.g. Prevotella bivia, Prevotella buccae, Prevotella corporis, Prevotella dentalis (Mitsuokella dentalis), Prevotella denticola, Prevotella disiens, Prevotella enoeca, Prevotella heparinolytica, Prevotella intermedia, Prevotella loeschii, Prevotella melaninogenica, Prevotella nigrescens, Prevotella oralis, Prevotella oris, Prevotella oulora, Prevotella tannerae, Prevotella venoralis and Prevotella zoogleoformans); Porphyromonas (e.g. Porphyromonas asaccharolytica, Porphyromonas cangingivalis, Porphyromonas canons, Porphyromonas cansulci, Porphyromonas catoniae, Porphyromonas circumdentaria, Porphyromonas crevioricanis, Porphyromonas endodontalis, Porphyromonas gingivalis, Porphyromonas gingivicanis, Porphyromonas levii and Porphyromonas macacae); Fusobacterium (e.g. F. gonadiaformans, F. mortiferum, F. naviforme, F. necrogenes, F. necrophorum necrophorum, F. necrophorum fundiliforme, F. nucleatum nucleatum, F. nucleatum fusiforme, F. nucleatum polymorphum, F. nucleatum vincentii, F. periodonticum, F. russii, F. ulcerans and F. varium); Chlamydia (e.g. Chlamydia trachomatis); Cryptosporidium (e.g. C. parvum, C. hominis, C. canis, C. felis, C. meleagridis and C. muris); Chlamydophila (e.g. Chlamydophila abortus (Chlamydia psittaci), Chlamydophila pneumoniae (Chlamydia pneumoniae) and Chlamydophila psittaci (Chlamydia psittaci)); Leuconostoc (e.g. Leuconostoc citreum, Leuconostoc cremoris, Leuconostoc dextranicum, Leuconostoc lactis, Leuconostoc mesenteroides and Leuconostoc pseudomesenteroides); Gemella (e.g. Gemella bergeri, Gemella haemolysans, Gemella morbillorum and Gemella sanguinis); Aeromonas (e.g. Aeromonas hydrophila, Aeromonas caviae and Aeromonas veronii biovar sobria); and Ureaplasma (e.g. Ureaplasma parvum and Ureaplasma urealyticum).

Preferably the combinations of the invention are synergistic against gram-positive or gram-negative bacteria selected from:

Gram-negatives: Enterobacteriaceae, Enterobacter spp, Pseudomonas spp, Acinetobacter spp, Shigella spp, Salmonella spp, Burkholderia stenotrophomonas, Citrobacter spp, Serratia spp, Proteus spp, Morganella spp, Providencia spp, Haemophilus spp, Aeromonas spp, Pasteurella spp, Brucella spp, Helicobacter spp, Campylobacter spp, Franciella tularensis, Legionella spp, Vibrio spp, Neisseria spp, Mycobacterium spp, Yersinia pestis, Rickettsia spp.

Gram-positives: Staphylococcus spp, Enterococcus spp, Streptococcus spp, Bacillus anthracis.

The Gram-negatives may, for example be: Enterobacteriaceae, such as Escherichia coli; Enterobacter (e.g. Enterobacter aerogenes, Enterobacter agglomerans and Enterobacter cloacae); Citrobacter (such as Citrob. freundii and Citrob. divernis); Pseudomonas (e.g. Ps. aeruginosa, Ps. maltophilia (Stenotrophomonas maltophilia), Ps. alcaligenes, Ps. chlororaphis, Ps. fluorescens, Ps. luteola. Ps. mendocina, Ps. monteilii, Ps. oryzihabitans, Ps. pertocinogena, Ps. pseudalcaligenes, Ps. putida and Ps. stutzeri); Yersinia (e.g. Yersinia enterocolitica, Yersinia pestis and Yersinia pseudotuberculosis); Helicobacter (e.g. Helicobacter pylori, Helicobacter cinaedi and Helicobacter fennelliae); Acinetobacter (e.g. A. baumanii, A. calcoaceticus, A. haemolyticus, A. johnsonii, A. junii, A. lwoffi and A. radioresistens); Morganella (e.g. Morganella morganii); Salmonella (Salmonella enterica and Salmonella typhi); Shigella (e.g. Shigella dysenteriae, Shigella flexneri, Shigella boydii and Shigella sonnei); Klebsiella (e.g. Klebs. pneumoniae, Klebs. oxytoca, Klebs. ornitholytica, Klebs. planticola, Klebs. ozaenae, Klebs. terrigena, Klebs. granulomatis (Calymmatobacterium granulomatis) and Klebs. rhinoscleromatis); Burkholderia stenotrophomonas; Francisella tularensis; Serratia (e.g. Serratia marcescens and Serratia liquifaciens); Proteus (e.g. Pr. mirabilis, Pr. rettgeri and Pr. vulgaris); Providencia (e.g. Providencia alcalifaciens, Providencia rettgeri and Providencia stuartii); Haemophilus (e.g. Haemophilus influenzae, Haemophilus ducreyi, Haemophilus aegyptius, Haemophilus parainfluenzae, Haemophilus haemolyticus and Haemophilus parahaemolyticus); Aeromonas (e.g. Aeromonas hydrophila, Aeromonas caviae and Aeromonas veronii biovar sobria); Pasteurella (e.g. Pasteurella aerogenes, Pasteurella bettyae, Pasteurella canis, Pasteurella dagmatis, Pasteurella gallinarum, Pasteurella haemolytica, Pasteurella multocida multocida, Pasteurella multocida gallicida, Pasteurella multocida septica, Pasteurella pneumotropica and Pasteurella stomatis); Brucella (e.g. Brucella abortus, Brucella canis, Brucella melintensis and Brucella suis); Campylobacter (e.g. Campylobacterjejuni, Campylobactercoli, Campylobacter lari and Campylobacter fetus); Legionella (e.g. Legionalla anisa, Legionalla birminghamensis, Legionalla bozemanii, Legionalla cincinnatiensis, Legionalla dumoffii, Legionalla feeleii, Legionalla gormanii, Legionalla hackeliae, Legionalla israelensis, Legionalla jordanis, Legionalla lansingensis, Legionalla Iongbeachae, Legionalla maceachernii, Legionalla micdadei, Legionalla oakridgensis, Legionalla pneumophila, Legionalla sainthelensi, Legionalla tucsonensis and Legionalla wadsworthii); Vibrio (e.g. Vibrio cholerae and Vibrio parahaemolyticus, Vibrio alginolyticus, Vibrio carchariae, Vibrio fluvialis, Vibrio furnissii, Vibrio hollisae, Vibrio metschnikovii, Vibrio mimicus and Vibrio vulnificus); Neisseria gonorrhoeae, Neisseria meningitidis, Neisseria cinerea, Neisseria elongata, Neisseria flavescens, Neisseria lactamica, Neisseria mucosa, Neisseria sicca, Neisseria subflava and Neisseria weaveri; Mycobacteria (e.g. Mycobacterium tuberculosis, Mycobacterium avium, Mycobacterium fortuitum, Mycobacterium marinum, Mycobacterium kansasii, Mycobacterium chelonae, Mycobacterium abscessus, Mycobacterium leprae, Mycobacterium smegmitis, Mycobacterium africanum, Mycobacterium alvei, Mycobacterium asiaticum, Mycobacterium aurum, Mycobacterium bohemicum, Mycobacterium bovis, Mycobacterium branderi, Mycobacterium brumae, Mycobacterium celatum, Mycobacterium chubense, Mycobacterium confluentis, Mycobacterium conspicuum, Mycobacterium cookii, Mycobacterium flavescens, Mycobacterium gadium, Mycobacterium gastri, Mycobacterium genavense, Mycobacterium gordonae, Mycobacterium goodii, Mycobacterium haemophilum, Mycobacterium hassicum, Mycobacterium intracellulare, Mycobacterium interjectum, Mycobacterium heidelberense, Mycobacterium lentiflavum, Mycobacterium malmoense, Mycobacterium microgenicum, Mycobacterium microti, Mycobacterium mucogenicum, Mycobacterium neoaurum, Mycobacterium nonchromogenicum, Mycobacterium peregrinum, Mycobacterium phlei, Mycobacterium scrofulaceum, Mycobacterium shimoidei, Mycobacterium simiae, Mycobacterium szulgai, Mycobacterium terrae, Mycobacterium thermoresistabile, Mycobacterium triplex, Mycobacterium triviale, Mycobacterium tusciae, Mycobacterium ulcerans, Mycobacterium vaccae, Mycobacterium wolinskyi and Mycobacterium xenopi); Rickettsia (e.g. Ricksettsia or Coxiella burnetii).

The Gram-positives may, for example, be Staphylococci (e.g. Staph. aureus, Staph. epidermidis, Staph. saprophyticus, Staph. auricularis, Staph. capitis capitis, Staph. c. ureolyticus, Staph. caprae, Staph. cohnii cohnii, Staph. c. urealyticus, Staph. equorum, Staph. gallinarum, Staph. haemolyticus, Staph. hominis hominis, Staph. h. novobiosepticius, Staph. hyicus, Staph. intermedius, Staph. lugdunensis, Staph. pasteuri, Staph. saccharolyticus, Staph. schleiferi schleiferi, Staph. s. coagulans, Staph. sciuri, Staph. simulans, Staph. warneri and Staph. xylosus); Streptococci (e.g.beta-haemolytic, pyogenic streptococci (such as Strept. agalactiae, Strept. canis, Strept. dysgalactiae dysgalactiae, Strept. dysgalactiae equisimilis, Strept. equi equi, Strept. equi zooepidemicus, Strept. iniae, Strept. porcinus and Strept. pyogenes), microaerophilic, pyogenic streptococci (Streptococcus “milleri”, such as Strept. anginosus, Strept. constellatus constellatus, Strept. constellatus pharyngidis and Strept. intermedius), oral streptococci of the “mitis” (alpha-haemolytic-Streptococcus “viridans”, such as Strept. mitis, Strept. oralis, Strept. sanguinis, Strept. cristatus, Strept. gordonii and Strept. parasanguinis), “salivarius” (non-haemolytic, such as Strept. salivarius and Strept. vestibularis) and “mutans” (tooth-surface streptococci, such as Strept. criceti, Strept. mutans, Strept. ratti and Strept. sobrinus) groups, Strept. acidominimus, Strept. bovis, Strept. faecalis, Strept. equinus, Strept. pneumoniae and Strept. suis, or Streptococci alternatively classified as Group A, B, C, D, E, G, L, P, U or V Streptococcus); Enterococci (e.g. Enterococcus avium, Enterococcus casseliflavus, Enterococcus cecorum, Enterococcus dispar, Enterococcus durans, Enterococcus faecalis, Enterococcus faecium, Enterococcus flavescens, Enterococcus gallinarum, Enterococcus hirae, Enterococcus malodoratus, Enterococcus mundtii, Enterococcus pseudoavium, Enterococcus raffinosus and Enterococcus solitarius); Bacillus anthracis.

The bacterial infections treated by the combinations described herein are Gram-negative or Gram-positive bacterial infections. Particular Gram-negative bacteria that may be treated using a combination of the invention include:

Enterobacteriaceae, such as Escherichia coli, Klebsiella (e.g. Klebs. pneumoniae and Klebs. oxytoca) and Proteus (e.g. Pr. mirabilis, Pr. rettgeri and Pr. vulgaris); Haemophilus influenzae; Mycobacteria, such as Mycobacterium tuberculosis; and Enterobacter (e.g. Enterobacter cloacae). Preferably, the bacteria are Enterobacteriaceae, such as Escherichia coli and Klebsiella (e.g. Klebs. pneumoniae and Klebs. oxytoca). Particularly preferred are Escherichia coli, and Klebs. pneumoniae (e.g. Klebs. pneumoniae subsp. pneumoniae).

The combination of the present invention is particularly beneficial in treating (multi)-drug-resistant ((M)DR) bacteria. With respect to Enterobacteriaceae, drug resistance most often builds up to carbapenemase i.e. carbapenemase-resistant strains and “extended spectrum β-lactamase” (ESBL) strains for example New Delhi Metallo-beta-lactamase-1 (NDM-1) resistant Klebs. Pneumoniae, and NDM-1 E. coli. The combination of the present invention is also particularly effective against carbapenemase-producing Enterobacteriaceae (CPE). Other drug-resistant strains may also be used, such as colistin-resistant strains, and carbapenemase-resistant strains of bacteria other than Enterobacteriaceae including carbapenem-resistant Acinetobacter and carbapenem-resistant Pseudomonas organisms carrying the blaKPC gene.

In various embodiments, the combination of the present invention is beneficial against the ESKAPE pathogens. These are six highly virulent and typically antibiotic resistant bacterial pathogens including Enterococcus faecium, Staphylococcus aureus, Klebsiella pneumoniae, Acinetobacter bacumannii, Pseudomonas aeruginosa, and Enterobacter spp. This group of Gram-positive and Gram-negative bacteria can evade or “escape” commonly used antibiotics due to their increasing multi-drug resistance. Accordingly the combination of the present invention is beneficial against (M)DR strains of the ESKAPE pathogens.

Advantageously, in various embodiments the combinations of the present invention may have a broader spectrum of activity than a monotherapy or combination of only two actives. In particular, various combinations may be efficacious against at least Acinetobacter, Pseudomonas and Enterobacteriaceae, which have been identified by the World Health Organisation as including multidrug resistant bacteria for which new antibiotics are critically needed.

It should be kept in mind that although a combination such as that claimed may initially be demonstrated to be functional in treating (M)DR strains, they can then be used in treating non-resistant strains. This is especially valuable in the context of the presently claimed combination where the primary therapy for Enterobacteriaceae, such as Escherichia coli, and Klebsiella (e.g. Klebs. pneumoniae and Klebs. oxytoca) are antimicrobial drugs that are expensive due to prevailing patent protection. The replacement of such “ethical” drugs by a combination of “generic” antibiotics is thought to be beneficial from a therapeutic perspective as well as financial/economic perspective in times where governments are seeking to reduce the cost of healthcare.

The combinations of the present invention may be used to treat infections associated with any of the above-mentioned bacterial organisms, and in particular they may be used for killing multiplying and/or clinically latent microorganisms associated with such an infection, e.g. an ESKAPE pathogen bacterial infection.

In various embodiments the combinations of the present invention are effective in treating infections caused by (1) Carbapenem-resistant E. coli, Klebsiella spp., Acinetobacter spp., Pseudomonas aeruginosa, Serratia spp, or Proteus, (2) MRSA, Vancomycin resistant Staphylococcus aureus (VRSA), Vancomycin resistant Enterococcus faecium (VRE), Clarithromycin resistant Helicobacter pylori, or Quinolone resistant Salmonella spp., or (3) penicillin resistant Streptococcus pneumoniae, Ampicillin resistant Haemophilus influenzae, or quinolone resistant Shigella spp.

Particular conditions which may be treated using the combination of the present invention include tuberculosis (e.g. pulmonary tuberculosis, non-pulmonary tuberculosis (such as tuberculosis lymph glands, genito-urinary tuberculosis, tuberculosis of bone and joints, tuberculosis meningitis) and miliary tuberculosis), anthrax, abscesses, acne vulgaris, actinomycosis, asthma, bacilliary dysentery, bacterial conjunctivitis, bacterial keratitis, bacterial vaginosis, botulism, Buruli ulcer, bone and joint infections, bronchitis (acute or chronic), brucellosis, burn wounds, cat scratch fever, cellulitis, chancroid, cholangitis, cholecystitis, cutaneous diphtheria, cystic fibrosis, cystitis, diffuse panbronchiolitis, diphtheria, dental caries, diseases of the upper respiratory tract, eczema, empyema, endocarditis, endometritis, enteric fever, enteritis, epididymitis, epiglottitis, erysipelis, erysipelas, erysipeloid, erythrasma, eye infections, furuncles, gardnerella vaginitis, gastrointestinal infections (gastroenteritis), genital infections, gingivitis, gonorrhoea, granuloma inguinale, Haverhill fever, infected burns, infections following dental operations, infections in the oral region, infections associated with prostheses, intraabdominal abscesses, Legionnaire's disease, leprosy, leptospirosis, listeriosis, liver abscesses, Lyme disease, lymphogranuloma venerium, mastitis, mastoiditis, meningitis and infections of the nervous system, mycetoma, nocardiosis (e.g. Madura foot), non-specific urethritis, opthalmia (e.g. opthalmia neonatorum), osteomyelitis, otitis (e.g. otitis externa and otitis media), orchitis, pancreatitis, paronychia, pelveoperitonitis, peritonitis, peritonitis with appendicitis, pharyngitis, phlegmons, pinta, plague, pleural effusion, pneumonia, postoperative wound infections, postoperative gas gangrene, prostatitis, pseudo-membranous colitis, psittacosis, pulmonary emphysema, pyelonephritis, pyoderma (e.g. impetigo), Q fever, rat-bite fever, reticulosis, ricin poisoning, Ritter's disease, salmonellosis, salpingitis, septic arthritis, septic infections, septicaemia, sinusitis, skin infections (e.g. skin granulomas, impetigo, folliculitis and furunculosis), syphilis, systemic infections, tonsillitis, toxic shock syndrome, trachoma, tularaemia, typhoid, typhus (e.g. epidemic typhus, murine typhus, scrub typhus and spotted fever), urethritis, wound infections, yaws, aspergillosis, candidiasis (e.g. oropharyngeal candidiasis, vaginal candidiasis or balanitis), cryptococcosis, favus, histoplasmosis, intertrigo, mucormycosis, tinea (e.g. tinea corporis, tinea capitis, tinea cruris, tinea pedis and tinea unguium), onychomycosis, pityriasis versicolor, ringworm and sporotrichosis; or infections with MSSA, MRSA, Staph. epidermidis, Strept. agalactiae, Strept. pyogenes, Escherichia coli, Klebs. pneumoniae, Klebs. oxytoca, Pr. mirabilis, Pr. rettgeri, Pr. vulgaris, Haemophilus influenzae, Enterococcus faecalis, Enterococcus faecium, Pseudomonas aeruginosa, and Acinetobacter baumanii.

Particular conditions which may be treated using the combination of the present invention also include those which are caused by Gram-negative bacteria such as abscesses, asthma, bacilliary dysentery, bacterial conjunctivitis, bacterial keratitis, bacterial vaginosis, bone and joint infections, bronchitis (acute or chronic), brucellosis, burn wounds, cat scratch fever, cellulitis, chancroid, cholangitis, cholecystitis, cystic fibrosis, cystitis, nephritis, diffuse panbronchiolitis, dental caries, diseases of the upper respiratory tract, empyema, endocarditis, endometritis, enteric fever, enteritis, epididymitis, epiglottitis, eye infections, furuncles, gardnerella vaginitis, gastrointestinal infections (gastroenteritis), genital infections, gingivitis, gonorrhoea, granuloma inguinale, Haverhill fever, infected burns, infections following dental operations, infections in the oral region, infections associated with prostheses, intraabdominal abscesses, Legionnaire's disease, leptospirosis, listeriosis, liver abscesses, Lyme disease, lymphogranuloma venerium, mastitis, mastoiditis, meningitis and infections of the nervous system, non-specific urethritis, opthalmia (e.g. opthalmia neonatorum), osteomyelitis, otitis (e.g. otitis externa and otitis media), orchitis, pancreatitis, paronychia, pelveoperitonitis, peritonitis, peritonitis with appendicitis, pharyngitis, pleural effusion, pneumonia, postoperative wound infections, postoperative gas gangrene, prostatitis, pseudo-membranous colitis, psittacosis, pyelonephritis, Q fever, Ritter's disease, salmonellosis, salpingitis, septic arthritis, septic infections, septicaemia, systemic infections, tonsillitis, trachoma, typhoid, urethritis, urinary tract infections, wound infections; or infections with, Escherichia coli, Klebs. pneumoniae, Klebs. oxytoca, Pr. mirabilis, Pr. rettgeri, Pr. vulgaris, Haemophilus influenzae, Enterococcus faecalis, Enterococcus faecium, and Enterobacter cloacae.

Preferably the combinations of the present invention are used to treat acute or complicated urinary tract infections, acute or complicated skin and soft tissue infections, intra-abdominal infections, upper respiratory tract infections, community-acquired pneumonia, hospital-acquired pneumonia, ventilator-associated pneumonia, or bloodstream infections.

It will be appreciated that references herein to “treatment” extend to prophylaxis as well as the treatment of established diseases or symptoms.

As used herein the term “pharmaceutically acceptable derivative” means: (a) pharmaceutically acceptable salts; (b) solvates (including hydrates) and/or (c) prodrugs (where appropriate).

Pharmaceutically acceptable salts of the compounds included in the combinations of the invention include suitable acid addition or base salts thereof. A review of suitable pharmaceutical salts may be found in Berge et al, J Pharm Sci, 66, 1-19 (1977).

Suitable acid addition salts include carboxylate salts (e.g. formate, acetate, trifluoroacetate, propionate, isobutyrate, heptanoate, decanoate, caprate, caprylate, stearate, acrylate, caproate, propiolate, ascorbate, citrate, glucuronate, glutamate, glycolate, α-hydroxybutyrate, lactate, tartrate, phenylacetate, mandelate, phenylpropionate, phenylbutyrate, benzoate, chlorobenzoate, methylbenzoate, hydroxybenzoate, methoxybenzoate, dinitrobenzoate, o-acetoxybenzoate, salicylate, nicotinate, isonicotinate, cinnamate, oxalate, malonate, succinate, suberate, sebacate, fumarate, malate, maleate, hydroxymaleate, hippurate, phthalate or terephthalate salts), halide salts (e.g. chloride, bromide or iodide salts), sulfonate salts (e.g. benzenesulfonate, methyl-, bromo- or chloro-benzenesulfonate, xylenesulfonate, methanesulfonate, ethanesulfonate, propanesulfonate, hydroxyethanesulfonate, 1- or 2-naphthalene-sulfonate or 1,5-naphthalenedisulfonate salts) or sulfate, pyrosulfate, bisulfate, sulfite, bisulfite, phosphate, monohydrogenphosphate, dihydrogenphosphate, metaphosphate, pyrophosphate or nitrate salts. Suitable base salts include metal salts, e.g. sodium, calcium, and amine salts.

For example, ceftazidime pentahydrate, colistin sulfate, polymyxin B sulfate, doxycycline hyclate (doxycycline hydrochloride hemiethanolate hemihydrate), doxycycline hydrochloride, doxycycline monohydrate, fosfomycin tromethamine, fosfomycin calcium, fosfomycin sodium, fosfomycin disodium, levofloxacin hemihydrate, meropenem trihydrate, rifampicin N-oxide, gentamicin sulfate hydrate, and gentamicin sulfate are commercially available from Sigma Aldrich. Other suppliers are also known in the art.

As used herein the term “prodrug” means the antimicrobial compound, wherein one or more groups have been modified such that the modification may be reversed upon administration to a human or mammalian subject. Such reversion is usually performed by an enzyme naturally present in such subject, though it is possible for a second agent to be administered together with such a prodrug in order to perform the reversion in vivo. Examples of such modifications include ester formation (for example, any of those described above), wherein the reversion may be carried out be an esterase etc. Other such systems will be well known to those skilled in the art.

Zidovudine is, for example, a prodrug that must be phosphorylated to its active 5′-triphosphate metabolite.

Polymyxin E or colistin is commercially available as a methanesulfonic acid derivative: colistimethate sodium or colistin sodium methanesulfonate (CMS). Colistimethate sodium is a prodrug. It is produced by the reaction of colistin with formaldehyde and sodium bisulfite, which leads to the addition of a sulfomethyl group to the primary amines of colistin. In aqueous solutions it undergoes hydrolysis to form a complex mixture of partially sulfomethylated derivatives and colistin.

The invention includes the use of these pharmaceutically acceptable derivatives and prodrugs. In particular, the invention includes the use of colistin and pharmaceutically acceptable derivatives thereof including colistin sulfate, colistimethate sodium and colistin sodium methanesulfonate.

The invention also includes where appropriate all enantiomers and tautomers of the compounds. The skilled person will recognise compounds that possess optical properties (one or more chiral carbon atoms) or tautomeric characteristics. The corresponding enantiomers and/or tautomers may be isolated or prepared by methods known in the art.

Some of the compounds included in the combinations of the invention may exist as stereoisomers and/or geometric isomers—e.g. they may possess one or more asymmetric and/or geometric centres and so may exist in two or more stereoisomeric and/or geometric forms. The present invention contemplates the use of all the individual stereoisomers and geometric isomers of those inhibitor agents, and mixtures thereof. The terms used in the claims encompass these forms, provided said forms retain the appropriate functional activity (though not necessarily to the same degree).

The present invention also includes all suitable isotopic variations of the compounds or pharmaceutically acceptable salts thereof. An isotopic variation or a pharmaceutically acceptable salt thereof is defined as one in which at least one atom is replaced by an atom having the same atomic number but an atomic mass different from the atomic mass usually found in nature. Examples of isotopes that can be incorporated include isotopes of hydrogen, carbon, nitrogen, oxygen, phosphorus, sulphur, fluorine and chlorine such as 2H, 3H, 13C, 14C, 15N, 170, 180, 31P, 32P, 35S, 18F and 36Cl, respectively. Certain isotopic variations, for example, those in which a radioactive isotope such as 3H or 14C is incorporated, are useful in drug and/or substrate tissue distribution studies. Tritiated, i.e., 3H, and carbon-14, i.e., 14C, isotopes are particularly preferred for their ease of preparation and detectability. Further, substitution with isotopes such as deuterium, i.e., 2H, may afford certain therapeutic advantages resulting from greater metabolic stability, for example, increased in vivo half-life or reduced dosage requirements and hence may be preferred in some circumstances. Isotopic variations can generally be prepared by conventional procedures using appropriate isotopic variations of suitable reagents.

The compounds for use in the combination of the present invention, including the pharmaceutically acceptable derivatives or prodrugs thereof, are commercially available and/or can be prepared by synthesis methods known in the art. Zidovudine, ceftazidime, polymyxin E, polymyxin B, doxycycline, fosfomycin, levofloxacin, meropenem, rifampicin, gentamicin, ceftazidime pentahydrate, colistin sulfate, colistimethate sodium, colistin sodium methanesulfonate, polymyxin B sulfate, doxycycline hyclate (doxycycline hydrochloride hemiethanolate hemihydrate), doxycycline hydrochloride, doxycycline monohydrate, fosfomycin tromethamine, fosfomycin calcium, fosfomycin sodium, fosfomycin disodium, levofloxacin hemihydrate, meropenem trihydrate, rifampicin N-oxide, gentamicin sulfate hydrate, and gentamicin sulfate are commercially available from Sigma Aldrich are for example available from Sigma-Aldrich®. Other commercial suppliers are known in the art.

Zidovudine is 1-[(2R, 4S, 5S)-4-Azido-5-(hydroxymethyl)oxolan-2-yl]-5-methylpyrimidine-2,4-dione, and is available by prescription under the trade name Retrovir®. It is also known as 3′-azido-3′-deoxythymidine or “AZT” and has the chemical structure:

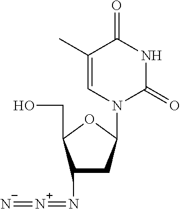 [SMILES: Cc1cn([C@H]2C[C@@H](N=[N+]=[N-])[C@@H](CO)O2)c(=O)[nH]c1=O]

Ceftazidime is sold under the brand name Fortaz among others, and is a third-generation cephalosporin antibiotic used in the treatment of a number of bacterial infections. It has the following chemical structure:

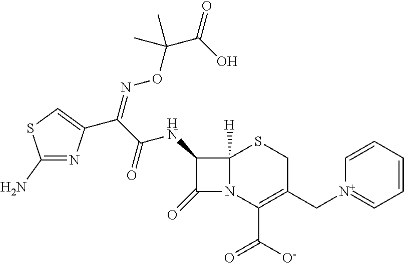 [SMILES: CC(C)(O/N=C(\C(=O)N[C@@H]1C(=O)N2C(C(=O)[O-])=C(C[n+]3ccccc3)CS[C@H]12)c1csc(N)n1)C(=O)O]

Polymyxin E is also known as colistin. It is an antibiotic medication used as a last-resort treatment for multidrug-resistant Gram-negative infections including pneumonia. These may involve bacteria such as Pseudomonas aeruginosa, Klebsiella pneumoniae, or Acinetobacter. It comes in two forms: colistimethate sodium can be injected into a vein, injected into a muscle, or inhaled, and colistin sulfate is mainly applied to the skin or taken by mouth. It has the following chemical structure:

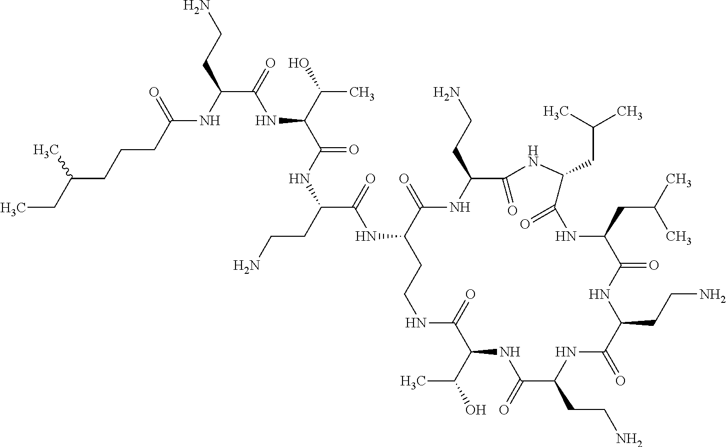 [SMILES: CCC(C)CCCC(=O)N[C@@H](CCN)C(=O)N[C@H](C(=O)N[C@@H](CCN)C(=O)N[C@H]1CCNC(=O)[C@H]([C@@H](C)O)NC(=O)[C@H](CCN)NC(=O)[C@H](CCN)NC(=O)[C@H](CC(C)C)NC(=O)[C@@H](CC(C)C)NC(=O)[C@H](CCN)NC1=O)[C@@H](C)O]

Polymyxin B, sold under the brand name Poly-Rx among others, is an antibiotic used to treat meningitis, pneumonia, sepsis, and urinary tract infections. It can be given by injection into a vein, muscle or cerebrospinal fluid, or inhaled. It has the following chemical structure:

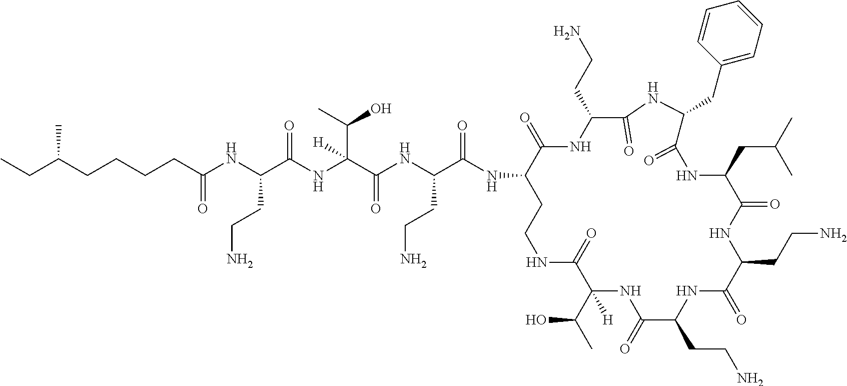 [SMILES: CC[C@H](C)CCCCC(=O)N[C@@H](CCN)C(=O)N[C@H](C(=O)N[C@@H](CCN)C(=O)N[C@H]1CCNC(=O)[C@H]([C@@H](C)O)NC(=O)[C@H](CCN)NC(=O)[C@H](CCN)NC(=O)[C@H](CC(C)C)NC(=O)[C@@H](Cc2ccccc2)NC(=O)[C@@H](CCN)NC1=O)[C@@H](C)O]

Doxycycline is a broad-spectrum tetracycline-class antibiotic used in the treatment of infections caused by bacteria and certain parasites. It is used to treat bacterial pneumonia, acne, chlamydia infections, Lyme disease, cholera, typhus, and syphilis. Doxycycline may be taken by mouth or by injection into a vein. It has the following chemical structure:

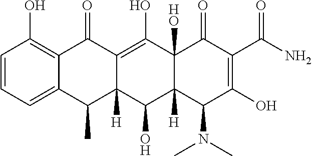 [SMILES: C[C@H]1c2cccc(O)c2C(=O)C2=C(O)[C@]3(O)C(=O)C(C(N)=O)=C(O)[C@@H](N(C)C)[C@@H]3[C@@H](O)[C@@H]21]

Fosfomycin, sold under the brand name Monurol among others, is an antibiotic primarily used to treat lower UTI. It is generally taken by mouth and has the following chemical structure:

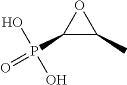

Levofloxacin, sold under the brand name Levaquin among others, is an antibiotic medication used to treat a number of bacterial infections including acute bacterial sinusitis, pneumonia, urinary tract infections, chronic prostatitis, and some types of gastroenteritis. It is available by mouth, intravenously, and in eye drop form. It is the (S)-isomer of ofloxacin and has the following chemical structure:

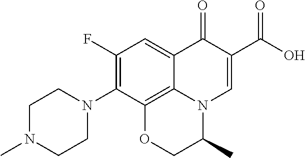 [SMILES: C[C@H]1COc2c(N3CCN(C)CC3)c(F)cc3c(=O)c(C(=O)O)cn1c23]

Meropenem, sold under the brand name Merrem among others, is an intravenous beta-lactam antibiotic used to treat a variety of bacterial infections. Some of these include meningitis, intra-abdominal infection, pneumonia, sepsis and anthrax. It is in the carbapenem family of medications and has the following chemical structure:

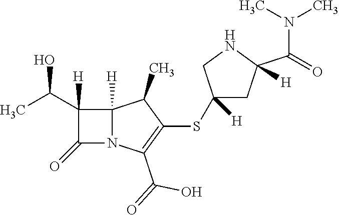 [SMILES: C[C@@H](O)[C@H]1C(=O)N2C(C(=O)O)=C(S[C@@H]3CN[C@H](C(=O)N(C)C)C3)[C@H](C)[C@H]12]

Rifampicin, also known as rifampin, is an ansamycin antibiotic used to treat several types of bacterial infections, including tuberculosis, Mycobacterium avium complex, leprosy, and Legionnaires' disease. Rifampicin may be given either by mouth or intravenously and has the following chemical structure:

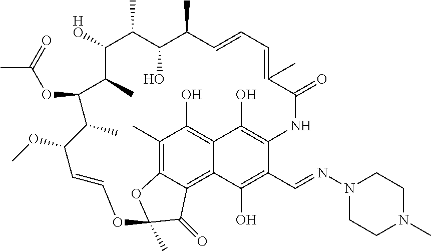 [SMILES: CO[C@H]1/C=C/O[C@@]2(C)Oc3c(C)c(O)c4c(O)c(c(/C=N/N5CCN(C)CC5)c(O)c4c3C2=O)NC(=O)/C(C)=C/C=C/[C@H](C)[C@H](O)[C@@H](C)[C@@H](O)[C@@H](C)[C@H](OC(C)=O)[C@@H]1C]

Gentamicin is an antibiotic used to treat several types of bacterial infections including bone infections, endocarditis, pelvic inflammatory disease, meningitis, pneumonia, urinary tract infections, and sepsis. It can be given intravenously, by intramuscular injection, or topically. It has the following chemical structure:

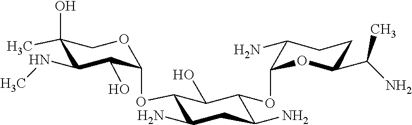

The synergistic combination of the present invention includes three antimicrobial agents. These agents are grouped in the appended claims so as to cover the exemplified combinations in the most efficient manner.

The first antimicrobial agent is selected from ceftazidime, polymyxin E, polymyxin B, and pharmaceutically acceptable derivatives thereof. The second antimicrobial agent is selected from zidovudine, doxycycline, fosfomycin, and pharmaceutically acceptable derivatives thereof; and the third antimicrobial agent is selected from levofloxacin, doxycycline, meropenem, rifampicin, gentamicin, polymyxin B, polymyxin E, and pharmaceutically acceptable derivatives thereof. The first, second and third antimicrobial agents are different from one another. The combination includes at least levofloxacin, doxycycline, rifampicin, fosfomycin or a pharmaceutically acceptable derivative thereof; provided the combination is not polymyxin E/B, zidovudine and rifampicin or ceftazidime, zidovudine and fosfomycin.

In various embodiments, the first antimicrobial agent is ceftazidime or a pharmaceutically acceptable derivative thereof. In some embodiments, the second antimicrobial agent is preferably zidovudine or a pharmaceutically acceptable derivative thereof. More preferably, ceftazidime and zidovudine, or pharmaceutically acceptable derivatives thereof, are combined with a third antimicrobial agent which is levofloxacin, doxycycline, rifampicin or a pharmaceutically acceptable derivative thereof. For example, the combination may be:
ceftazidime or a pharmaceutically acceptable derivative thereof; zidovudine or a pharmaceutically acceptable derivative thereof; and levofloxacin or a pharmaceutically acceptable derivative thereof;
ceftazidime or a pharmaceutically acceptable derivative thereof; zidovudine or a pharmaceutically acceptable derivative thereof; and doxycycline or a pharmaceutically acceptable derivative thereof; or
ceftazidime or a pharmaceutically acceptable derivative thereof; zidovudine or a pharmaceutically acceptable derivative thereof; and rifampicin or a pharmaceutically acceptable derivative thereof.

In various embodiments, the first antimicrobial agent is ceftazidime or a pharmaceutically acceptable derivative thereof. In some embodiments, the second antimicrobial agent is preferably doxycycline, fosfomycin, or a pharmaceutically acceptable derivative thereof. More preferably, ceftazidime and doxycycline or fosfomycin, or pharmaceutically acceptable derivatives thereof, are combined with a third antimicrobial agent which is levofloxacin, doxycycline (when the second antimicrobial is fosfomycin or a pharmaceutically acceptable derivative thereof), meropenem, rifampicin, gentamicin, polymyxin B, polymyxin E, or a pharmaceutically acceptable derivative thereof. For example, the combination may be:
ceftazidime or a pharmaceutically acceptable derivative thereof; doxycycline or a pharmaceutically acceptable derivative thereof; and levofloxacin or a pharmaceutically acceptable derivative thereof;
ceftazidime or a pharmaceutically acceptable derivative thereof; doxycycline or a pharmaceutically acceptable derivative thereof; and meropenem or a pharmaceutically acceptable derivative thereof;
ceftazidime or a pharmaceutically acceptable derivative thereof; doxycycline or a pharmaceutically acceptable derivative thereof; and rifampicin or a pharmaceutically acceptable derivative thereof;
ceftazidime or a pharmaceutically acceptable derivative thereof; doxycycline or a pharmaceutically acceptable derivative thereof; and gentamicin or a pharmaceutically acceptable derivative thereof;
ceftazidime or a pharmaceutically acceptable derivative thereof; doxycycline or a pharmaceutically acceptable derivative thereof; polymyxin E/B or a pharmaceutically acceptable derivative thereof;
ceftazidime or a pharmaceutically acceptable derivative thereof; fosfomycin or a pharmaceutically acceptable derivative thereof; and levofloxacin or a pharmaceutically acceptable derivative thereof;
ceftazidime or a pharmaceutically acceptable derivative thereof; fosfomycin or a pharmaceutically acceptable derivative thereof; and doxycycline or a pharmaceutically acceptable derivative thereof;
ceftazidime or a pharmaceutically acceptable derivative thereof; fosfomycin or a pharmaceutically acceptable derivative thereof; and meropenem or a pharmaceutically acceptable derivative thereof;
ceftazidime or a pharmaceutically acceptable derivative thereof; fosfomycin or a pharmaceutically acceptable derivative thereof; and rifampicin or a pharmaceutically acceptable derivative thereof;
ceftazidime or a pharmaceutically acceptable derivative thereof; fosfomycin or a pharmaceutically acceptable derivative thereof; and gentamicin or a pharmaceutically acceptable derivative thereof; or
ceftazidime or a pharmaceutically acceptable derivative thereof; fosfomycin or a pharmaceutically acceptable derivative thereof; polymyxin E/B or a pharmaceutically acceptable derivative thereof.

In various embodiments, the first antimicrobial agent is polymyxin E or a pharmaceutically acceptable derivative thereof and the second antimicrobial agent is doxycycline, fosfomycin, or a pharmaceutically acceptable derivative thereof. In such embodiments, the third antimicrobial agent is preferably selected from levofloxacin, doxycycline (when the second antimicrobial is fosfomycin), meropenem, rifampicin, gentamicin, or a pharmaceutically acceptable derivative thereof. For example, the combination may be:
polymyxin E or a pharmaceutically acceptable derivative thereof; doxycycline or a pharmaceutically acceptable derivative thereof; and levofloxacin or a pharmaceutically acceptable derivative thereof;
polymyxin E or a pharmaceutically acceptable derivative thereof; doxycycline or a pharmaceutically acceptable derivative thereof; and meropenem or a pharmaceutically acceptable derivative thereof;
polymyxin E or a pharmaceutically acceptable derivative thereof; doxycycline or a pharmaceutically acceptable derivative thereof; and rifampicin or a pharmaceutically acceptable derivative thereof;
polymyxin E or a pharmaceutically acceptable derivative thereof; doxycycline or a pharmaceutically acceptable derivative thereof; and gentamicin or a pharmaceutically acceptable derivative thereof;
polymyxin E or a pharmaceutically acceptable derivative thereof; fosfomycin or a pharmaceutically acceptable derivative thereof; and levofloxacin or a pharmaceutically acceptable derivative thereof;
polymyxin E or a pharmaceutically acceptable derivative thereof; fosfomycin or a pharmaceutically acceptable derivative thereof; and doxycycline or a pharmaceutically acceptable derivative thereof;
polymyxin E or a pharmaceutically acceptable derivative thereof; fosfomycin or a pharmaceutically acceptable derivative thereof; and meropenem or a pharmaceutically acceptable derivative thereof;
polymyxin E or a pharmaceutically acceptable derivative thereof; fosfomycin or a pharmaceutically acceptable derivative thereof; and rifampicin or a pharmaceutically acceptable derivative thereof; or
polymyxin E or a pharmaceutically acceptable derivative thereof; fosfomycin or a pharmaceutically acceptable derivative thereof; and gentamicin or a pharmaceutically acceptable derivative thereof.

In various embodiments, the first antimicrobial agent is polymyxin E/B or a pharmaceutically acceptable derivative thereof, the second antimicrobial agent is zidovudine or a pharmaceutically acceptable derivative thereof, and the third antimicrobial agent is fosfomycin or a pharmaceutically acceptable derivative thereof.

The present invention also provides an antimicrobial combination with three antimicrobial agents where the first antimicrobial agent is ceftazidime or a pharmaceutically acceptable derivative thereof, the second antimicrobial agent is zidovudine or a pharmaceutically acceptable derivative thereof, and the third antimicrobial agent is polymyxin E or a pharmaceutically acceptable derivative thereof.

The combinations of the present invention may be grouped by one or more common antimicrobial agent. In one embodiment, the combinations include ceftazidime or a pharmaceutically acceptable derivative thereof as the first antimicrobial agent and zidovudine or a pharmaceutically acceptable derivative thereof as the second antimicrobial agent, provided that the third antimicrobial agent is not fosfomycin. For example, one aspect of the present invention provides an antimicrobial combination comprising three antimicrobial agents, wherein the first antimicrobial agent is ceftazidime or a pharmaceutically acceptable derivative thereof; the second antimicrobial agent is zidovudine or a pharmaceutically acceptable derivative thereof; and the third antimicrobial agent is meropenem or a pharmaceutically acceptable derivative thereof.

In one embodiment, the combinations include ceftazidime or a pharmaceutically acceptable derivative thereof as the first antimicrobial agent and levofloxacin or a pharmaceutically acceptable derivative thereof as the second antimicrobial agent; and the third antimicrobial agent is meropenem or a pharmaceutically acceptable derivative thereof.

In another embodiment, the combinations include ceftazidime or a pharmaceutically acceptable derivative thereof as the first antimicrobial agent, fosfomycin/doxycycline or a pharmaceutically acceptable derivative thereof as the second antimicrobial agent, and fosfomycin/doxycycline or a pharmaceutically acceptable derivative thereof as the third antimicrobial agent.

In another embodiment the combinations include ceftazidime or a pharmaceutically acceptable derivative thereof as the first antimicrobial agent and doxycycline or a pharmaceutically acceptable derivative thereof as the second antimicrobial agent.

In another embodiment the combinations include polymyxin E/B or a pharmaceutically acceptable derivative thereof as the first antimicrobial agent and fosfomycin, doxycycline, or a pharmaceutically acceptable derivative thereof as the second antimicrobial agent.

In various embodiments, any of the combinations disclosed herein may include a fourth antimicrobial agent. In preferred embodiments, the fourth antimicrobial agent is a carbapenem or a pharmaceutically acceptable derivative thereof. More preferably the fourth antimicrobial agent is selected from the group consisting of imipenem, meropenem, ertapenem, doripenem, panipenem, biapenem, razupenem, tebipenem, lenapenem, tomopenem and thienpenem or a pharmaceutically derivative thereof, e.g. meropenem trihydrate. Even more preferably, the fourth antimicrobial agent is meropenem or a pharmaceutically acceptable derivative thereof.

In one embodiment, the first antimicrobial agent is polymyxin E/B or a pharmaceutically acceptable derivative thereof, the second antimicrobial agent is zidovudine or a pharmaceutically acceptable derivative thereof, the third antimicrobial agent is fosfomycin or a pharmaceutically acceptable derivative thereof, and the fourth antimicrobial agent is a carbapenem or a pharmaceutically acceptable derivative thereof, preferably a carbapenem selected from the group consisting of imipenem, meropenem, ertapenem, doripenem, panipenem, biapenem, razupenem, tebipenem, lenapenem, tomopenem and thienpenem or a pharmaceutically derivative thereof, e.g. meropenem trihydrate; more preferably meropenem or a pharmaceutically acceptable derivative thereof.

In one embodiment, the first antimicrobial agent is ceftazidime or a pharmaceutically acceptable derivative thereof; the second antimicrobial agent is zidovudine or a pharmaceutically acceptable derivative thereof; the third antimicrobial agent is polymyxin E/B or a pharmaceutically acceptable derivative thereof, and the fourth antimicrobial agent is a carbapenem or a pharmaceutically acceptable derivative thereof, preferably a carbapenem selected from the group consisting of imipenem, meropenem, ertapenem, doripenem, panipenem, biapenem, razupenem, tebipenem, lenapenem, tomopenem and thienpenem or a pharmaceutically derivative thereof, e.g. meropenem trihydrate; more preferably meropenem or a pharmaceutically acceptable derivative thereof.

In one embodiment, the first antimicrobial agent is ceftazidime or a pharmaceutically acceptable derivative thereof; the second antimicrobial agent is zidovudine or a pharmaceutically acceptable derivative thereof; the third antimicrobial agent is polymyxin E/B or a pharmaceutically acceptable derivative thereof; and the fourth antimicrobial agent is a carbapanem or a pharmaceutically acceptable derivative thereof, preferably a carbapenem selected from the group consisting of imipenem, meropenem, ertapenem, doripenem, panipenem, biapenem, razupenem, tebipenem, lenapenem, tomopenem and thienpenem or a pharmaceutically derivative thereof, e.g. meropenem trihydrate; more preferably meropenem or a pharmaceutically acceptable derivative thereof.

Compounds for use according to the invention may be administered as the raw material but are preferably provided in the form of pharmaceutical compositions. The compounds may be used either as separate formulations or as a single combined formulation. When combined in the same formulation it will be appreciated that the two compounds must be stable and compatible with each other and the other components of the formulation.

Formulations of the invention include those suitable for oral, parenteral (including subcutaneous e.g. by injection or by depot tablet, intrathecal, intramuscular e.g. by depot and intravenous), and rectal or in a form suitable for administration by inhalation or insufflation administration. The most suitable route of administration may depend upon the condition and disorder of the patient. Preferably, the compositions of the invention are formulated for oral administration.

The formulations may conveniently be presented in unit dosage form and may be prepared by any of the methods well known in the art of pharmacy e.g. as described in “Remington: The Science and Practice of Pharmacy”, Lippincott Williams and Wilkins, 21^{st} Edition, (2005). Suitable methods include the step of bringing into association to active ingredients with a carrier which constitutes one or more excipients. In general, formulations are prepared by uniformly and intimately bringing into association the active ingredients with liquid carriers or finely divided solid carriers or both and then, if necessary, shaping the product into the desired formulation. It will be appreciated that when the two active ingredients are administered independently, each may be administered by a different means.

When formulated with excipients, the active ingredients may be present in a concentration from 0.1 to 99.5% (such as from 0.5 to 95%) by weight of the total mixture; conveniently from 30 to 95% for tablets and capsules and 0.01 to 50% (such as from 3 to 50%) for liquid preparations.

The concentration of each antimicrobial agent in the synergistic combination is equal to or less than the minimum inhibitory concentration in monotherapy for the bacteria against which the combination is being used (i.e. MICₘₒₙₒ). Reference herein to “MIC” should therefore be understood as MICₘₒₙₒ unless otherwise specified. Preferably the concentration of at least one of the antimicrobial agents in the synergistic combination is less than MICₘₒₙₒ, more preferably the concentration of at least two of the antimicrobial agents in the synergistic combination is less than MICₘₒₙₒ. The use of such concentrations is advantageous because it avoids toxicity issues and the reduces the possibility of antimicrobial resistance developing vs one or more of the agents in the combination.

In various embodiments, the concentration of ceftazidime is 1×MIC or less for the bacteria against which the combination is being used, the MIC being the minimum inhibitory concentration of ceftazidime when used alone against said bacteria, i.e. MICₘₒₙₒ. Preferably the concentration of ceftazidime may be 0.5×MICₘₒₙₒ or less for the bacteria against which the combination is being used. More preferably the concentration of ceftazidime is 0.25×MICₘₒₙₒ or less for the bacteria against which the combination is being used. Most preferably, the concentration of ceftazidime is 0.125×MICₘₒₙₒ or less for the bacteria against which the combination is being used. In some embodiments, the concentration of ceftazidime is as low as 0.0625×MICₘₒₙₒ—equivalent to 1/16^{th} MICₘₒₙₒ—for the bacteria against which the combination is being used. Indeed, the unexpectedly enhanced potencies obtained by the combinations of the present invention are such that in some embodiments such as those comprising four antimicrobial agents, the concentration of e.g. ceftazidime may be as low as 1/512^{th} MICₘₒₙₒ for the bacteria against which the combination is being used.

As an example, the ceftazidime concentration may be about 32 mg/L or less in a combination showing synergy against ESBL E. coli. In a preferred embodiment the ceftazidime concentration may be about 0.5 to about 32 mg/L in a combination showing synergy against ESBL E. coli. More preferably the ceftazidime concentration may be about 0.5 to about 16 mg/L in a combination showing synergy against ESBL E. coli. Most preferably the ceftazidime concentration may be about 0.5 to about 4 mg/L in a combination showing synergy against ESBL E. coli.

As a further example, the ceftazidime concentration may be about 4 mg/L or less in a combination showing synergy against K. pneumoniae CPE. In a preferred embodiment, the ceftazidime concentration may be about 0.25 to about 4 mg/L in a combination showing synergy against K. pneumoniae CPE. More preferably the ceftazidime concentration may be about 0.25 to about 2 mg/L in a combination showing synergy against K. pneumoniae CPE.

As a further example, the ceftazidime concentration may be about 512 mg/L or less in a combination showing synergy against P. aeruginosa. In a preferred embodiment, the ceftazidime concentration may be about 0.015625 to about 512 mg/L in a combination showing synergy against P. aeruginosa. More preferably, the ceftazidime concentration may be about 0.5 to about 4 mg/L in a combination showing synergy against P. aeruginosa.

As a further example, the ceftazidime concentration may be about 512 mg/L or less in a combination showing synergy against A. baumanii. In a preferred embodiment, the ceftazidime concentration may be about 32 to about 512 mg/L in a combination showing synergy against A. baumanii. More preferably, the ceftazidime concentration may be about 32 to about 128 mg/L.

As a further example, ceftazidime concentration may be about 32 mg/L or less in a combination showing synergy against MRSA. In a preferred embodiment, the ceftazidime concentration may be about 1 to about 32 mg/L in a combination showing synergy against MRSA.

In various embodiments, the concentration of zidovudine is 1×MICₘₒₙₒ or less for the bacteria against which the combination is being used. Preferably the concentration of zidovudine may be 0.5×MICₘₒₙₒ or less for the bacteria against which the combination is being used. More preferably the concentration of zidovudine is 0.25×MICₘₒₙₒ or less for the bacteria against which the combination is being used. Most preferably, the concentration of zidovudine is 0.125×MICₘₒₙₒ or less for the bacteria against which the combination is being used.

As an example, the zidovudine concentration may be about 1 mg/L or less in a combination showing synergy against ESBL E. coli, preferably about 0.03 to about 1 mg/L, more preferably about 0.125 to about 1 mg/L.

As a further example, the zidovudine concentration may be about 512 mg/L or less in a combination showing synergy against P. aeruginosa, preferably about 128 mg/L or less, or more preferably about 64 mg/L or less in a combination showing synergy against P. aeruginosa. For instance, in various embodiments the zidovudine concentration may be 0.5 to about 512 mg/L, about 1 to about 512 mg/L, about 2 to about 512 mg/L or about 8 to about 512 mg/L in a combination showing synergy against P. aeruginosa. In further embodiments, the zidovudine concentration may be 0.5, 1, 2, or 8 mg/L to about 128, more preferably to about 64 mg/L, in a combination showing synergy against P. aeruginosa.

As a further example, the zidovudine concentration may be about 512 mg/L or less in a combination showing synergy against A. baumanii, preferably about 128 mg/L or less. In various embodiments, the zidovudine concentration may be about 0.5 to about 512 mg/L in a combination showing synergy against A. baumanii, preferably about 2 to about 512 mg/L, about 2 to about 128 mg/L or about 4 to about 128 mg/L.

As will be understood by the person skilled in the art, the concentration ranges for zidovudine as second antimicrobial agent may be combined with the concentration ranges for ceftazidime as first antimicrobial agent and further with the concentration ranges below for each of levofloxacin, doxycycline, and rifampicin as the third antimicrobial agent as defined below. The skilled person would also understand that all of the concentration ranges herein apply to pharmaceutically acceptable derivatives of the named compounds.

In various embodiments, ceftazidime is used at a concentration of about 0.5 to about 32 mg/L and zidovudine is used at a concentration of about 0.03 to about 1 mg/L in a combination showing synergy against ESBL E. coli. Preferably the ceftazidime concentration may be about 0.5 to about 16 mg/L and the zidovudine concentration may be about 0.03 to about 1 mg/L in a combination showing synergy against ESBL E. coli. Most preferably the ceftazidime concentration may be about 0.5 to about 4 mg/L and the zidovudine concentration may be about 0.03 to about 1 mg/L in a combination showing synergy against ESBL E. coli.

In various embodiments, ceftazidime is used at a concentration of about 0.015625 to about 512 mg/L and zidovudine is used at a concentration of about 0.5 to about 512 mg/L in a combination showing synergy against P. aeruginosa. Preferably the ceftazidime concentration may be about 0.5 to about 512 mg/L and the zidovudine concentration may be about 2 to about 512 mg/L in a combination showing synergy against P. aeruginosa. More preferably the ceftazidime concentration may be about 0.5 to about 4 mg/L and the zidovudine concentration may be about 8 to about 128 mg/L, more preferably about 64 mg/L, in a combination showing synergy against P. aeruginosa.

In various embodiments, ceftazidime is used at a concentration of about 32 to about 512 mg/L and zidovudine is used at a concentration of about 0.5 to about 512 mg/L in a combination showing synergy against A. baumanii. Preferably the ceftazidime concentration may be about 32 to about 512 mg/L and the zidovudine concentration may be about 0.5, 2, or 4 mg/L to about 512 mg/L in a combination showing synergy against A. baumanii. More preferably, the ceftazidime concentration may be about 32 to about 128 mg/L and the zidovudine concentration may be about 0.5, about 2 or about 4 mg/L to about 128 mg/L in a combination showing synergy against A. baumanii.

In various embodiments, the concentration of levofloxacin is 1×MICₘₒₙₒ or less for the bacteria against which the combination is being used. Preferably the concentration of levofloxacin may be 0.5×MICₘₒₙₒ or less for the bacteria against which the combination is being used. More preferably the concentration of levofloxacin may be 0.25×MICₘₒₙₒ or less for the bacteria against which the combination is being used. Most preferably, the concentration of levofloxacin is 0.125×MICₘₒₙₒ or less for the bacteria against which the combination is being used.

As an example, the levofloxacin concentration may be about 8 mg/L or less in a combination showing synergy against ESBL E. coli, preferably about 0.25 to about 8 mg/L, more preferably about 0.25 to about 4 mg/L.

As a further example, the levofloxacin concentration may be about 1 mg/L or less in a combination showing synergy against MRSA, preferably about 0.125 to about 1 mg/L, more preferably about 0.25 to about 1 mg/L.

As noted above, the concentration ranges for levofloxacin may be combined with those for each of ceftazidime and zidovudine. In various embodiments, ceftazidime is used at a concentration of about 0.5 to about 32 mg/L, zidovudine is used at a concentration of about 0.03 to about 1 mg/L and levofloxacin is used at a concentration of about 0.25 to about 8 mg/L in a combination showing synergy against ESBL E. coli. Preferably the ceftazidime concentration may be about 0.5 to about 16 mg/L, the zidovudine concentration may be about 0.03 to about 1 mg/L and the levofloxacin concentration may be about 1 to about 8 mg/L in a combination showing synergy against ESBL E. coli. Most preferably the ceftazidime concentration may be about 0.5 to about 4 mg/L, the zidovudine concentration may be about 0.03 to about 1 mg/L and the levofloxacin concentration may be about 4 to about 8 mg/L, most preferably about 8 mg/L in a combination showing synergy against ESBL E. coli.

In various embodiments, ceftazidime is used at a concentration of about 0.5 to about 4 mg/L, zidovudine is used at a concentration of about 0.25 to about 1 mg/L and levofloxacin is used at a concentration of about 0.25 to about 8 mg/L in a combination showing synergy against ESBL E. coli. Preferably, ceftazidime may be used at a concentration of about 0.5 to about 4 mg/L, zidovudine may be used at a concentration of about 0.5 to about 1 mg/L and levofloxacin may be used at a concentration of about 1 to about 8 mg/L in a combination showing synergy against ESBL E. coli.

The concentrations of each of ceftazidime, zidovudine, and levofloxacin may also be expressed as factors of MICₘₒₙₒ. For example, ceftazidime may be used at a concentration of 1×MICₘₒₙₒ or less, zidovudine may be used at a concentration of 1×MICₘₒₙₒ or less, and levofloxacin may be used at a concentration of 1×MICₘₒₙₒ or less. Preferably, ceftazidime may be used at a concentration of 1×MICₘₒₙₒ or less, levofloxacin may be used at a concentration of 1×MICₘₒₙₒ or less and zidovudine may be used at a concentration of 0.5 to 1×MICₘₒₙₒ. Alternatively, ceftazidime may be used at a concentration of 0.5×MICₘₒₙₒ or less, levofloxacin may be used at a concentration of 1×MICₘₒₙₒ, and zidovudine is used at a concentration of 1×MICₘₒₙₒ or less.

In various embodiments the concentration of doxycycline is 1×MICₘₒₙₒ or less for the bacteria against which the combination is being used. Preferably the concentration of doxycycline may be 0.5×MICₘₒₙₒ or less for the bacteria against which the combination is being used. More preferably the concentration of doxycycline may be 0.25×MICₘₒₙₒ or less for the bacteria against which the combination is being used. Most preferably the concentration of doxycycline may be 0.125×MICₘₒₙₒ or less for the bacteria against which the combination is being used.

As an example, the doxycycline concentration may be about 1 mg/L or less in a combination showing synergy against ESBL E. coli, preferably about 0.03 to about 1 mg/L, more preferably about 0.03 to about 0.5 mg/L.

As noted above, the concentration ranges for doxycycline may be combined with those for each of ceftazidime and zidovudine. The concentration ranges for doxycycline may also be combined with those for each of polymyxin E/B and levofloxacin/meropenem/rifampicin/gentamicin, or for each of polymyxin E/B and zidovudine.

In various embodiments, ceftazidime is used at a concentration of about 0.5 to about 32 mg/L, zidovudine is used at a concentration of about 0.03 to about 1 mg/L and doxycycline is used at a concentration of about 0.03 to about 2 mg/L in a combination showing synergy against ESBL E. coli. Preferably the ceftazidime concentration may be about 0.5 to about 16 mg/L, the zidovudine concentration may be about 0.03 to about 1 mg/L and the doxycycline concentration may be about 0.06 to about 1 mg/L in a combination showing synergy against ESBL E. coli. Most preferably the ceftazidime concentration may be about 0.5 to about 4 mg/L, the zidovudine concentration may be about 0.03 to about 1 mg/L and the doxycycline concentration may be about 0.5 to about 1 mg/L, most preferably about 1 mg/L in a combination showing synergy against ESBL E. coli.

In various embodiments ceftazidime is used at a concentration of about 0.5 to about 4 mg/L, zidovudine is used at a concentration of about 0.25 to about 1 mg/L and doxycycline is used at a concentration of about 0.03 to about 1 mg/L in a combination showing synergy against ESBL E. coli. Preferably, ceftazidime may be used at a concentration of about 0.5 to about 4 mg/L, zidovudine may be used at a concentration of about 0.5 to about 1 mg/L and doxycycline is used at a concentration of about 0.06 to about 1 mg/L, more preferably about 0.5 to about 1 mg/L in a combination showing synergy against ESBL E. coli.

The concentrations of each of ceftazidime, zidovudine, and doxycycline may also be expressed as factors of MICₘₒₙₒ. For example, ceftazidime may be used at a concentration of 1×MICₘₒₙₒ or less, doxycycline may be used at a concentration of 1×MICₘₒₙₒ or less and zidovudine may be used at a concentration of 1×MICₘₒₙₒ or less. Preferably, ceftazidime may be used at a concentration of 1×MICₘₒₙₒ or less, doxycycline may be used at a concentration of 1×MICₘₒₙₒ or less and zidovudine may be used at a concentration of 0.25 to 1×MICₘₒₙₒ. Alternatively, ceftazidime may be used at a concentration of 0.5×MICₘₒₙₒ or less, doxycycline may be used at a concentration of 1×MICₘₒₙₒ, and zidovudine is used at a concentration of 1×MICₘₒₙₒ or less.

The concentration ranges for doxycycline may alternatively be combined with those for each of ceftazidime and levofloxacin. In various embodiments, ceftazidime is used at a concentration of about 0.5 to about 32 mg/L, doxycycline is used at a concentration of about 0.0625 to about 1 mg/L and levofloxacin is used at a concentration of about 0.25 to about 8 mg/L in a combination showing synergy against ESBL E. coli. Preferably the ceftazidime concentration may be about 0.5 to about 16 mg/L, the doxycycline concentration may be about 0.0625 to about 1 mg/L and the levofloxacin concentration may be about 2 to about 4 mg/L in a combination showing synergy against ESBL E. coli. Alternatively the ceftazidime concentration may be about 0.5 to about 16 mg/L, the doxycycline concentration may be about 0.0625 to about 0.5 mg/L and the levofloxacin concentration may be about 0.5 to about 4 mg/L in a combination showing synergy against ESBL E. coli. Most preferably the ceftazidime concentration may be about 0.5 to about 4 mg/L, the doxycycline concentration may be about 0.125 to about 1 mg/L and the levofloxacin concentration may be about 2 to about 8 mg/L.

The concentrations of each of ceftazidime, doxycycline and levofloxacin may also be expressed as factors of MICₘₒₙₒ. For example, ceftazidime may be used at a concentration of 1×MICₘₒₙₒ or less, doxycycline may be used at a concentration of 1×MICₘₒₙₒ or less and levofloxacin may be used at a concentration of 1×MICₘₒₙₒ or less. Preferably, ceftazidime may be used at a concentration of 1×MICₘₒₙₒ or less, doxycycline may be used at a concentration of 1×MICₘₒₙₒ or less and levofloxacin may be used at a concentration of 1×MICₘₒₙₒ. More preferably, ceftazidime may be used at a concentration of 0.5×MICₘₒₙₒ or less, doxycycline may be used at a concentration of 1×MICₘₒₙₒ, and levofloxacin is used at a concentration of 1×MICₘₒₙₒ or less.

As a further example, the doxycycline concentration may be 0.5 mg/L or less in a combination showing synergy against K. pneumoniae CPE, preferably 0.03 to 0.5 mg/L, more preferably 0.03 to 0.25 mg/L.

In various embodiments the concentration of rifampicin may be 1×MICₘₒₙₒ or less for the bacteria against which the combination is being used. Preferably the concentration of rifampicin may be 0.5×MICₘₒₙₒ or less for the bacteria against which the combination is being used. More preferably the concentration of rifampicin may be 0.25×MICₘₒₙₒ or less for the bacteria against which the combination is being used. Most preferably the concentration of rifampicin may be 0.125×MICₘₒₙₒ or less for the bacteria against which the combination is being used.

As an example, the rifampicin concentration may be about 4 mg/L or less in a combination showing synergy against ESBL E. coli, preferably about 0.125 to about 4 mg/L, more preferably about 0.25 to about 4 mg/L.

The concentration ranges for rifampicin may be combined with those for each of ceftazidime or polymyxin E/B and zidovudine, doxycycline or fosfomycin.

In various embodiments, ceftazidime is used at a concentration of about 0.5 to about 32 mg/L, zidovudine is used at a concentration of about 0.03 to about 1 mg/L and rifampicin is used at a concentration of about 0.125 to about 4 mg/L in a combination showing synergy against ESBL E. coli. Preferably the ceftazidime concentration may be about 0.5 to about 16 mg/L, the zidovudine concentration may be about 0.03 to about 1 mg/L and the rifampicin concentration may be about 0.5 to about 4 mg/L in a combination showing synergy against ESBL E. coli. Most preferably the ceftazidime concentration may be about 0.5 to about 4 mg/L, the zidovudine concentration may be about 0.03 to about 1 mg/L and the rifampicin concentration may be about 0.5 to about 4 mg/L, most preferably about 1 mg/L to about 4 mg/L in a combination showing synergy against ESBL E. coli.

In various embodiments ceftazidime is used at a concentration of about 0.5 to about 4 mg/L, zidovudine is used at a concentration of about 0.125 to about 1 mg/L and rifampciin is used at a concentration of about 0.5 to about 4 mg/L in a combination showing synergy against ESBL E. coli. Preferably, ceftazidime may be used at a concentration of about 0.5 to about 4 mg/L, zidovudine may be used at a concentration of about 0.125 to about 1 mg/L and rifampicin is used at a concentration of about 1 to about 4 mg/in a combination showing synergy against ESBL E. coli.

The concentrations of each of ceftazidime, zidovudine and rifampicin may also be expressed as factors of MICₘₒₙₒ. For example, ceftazidime may be used at a concentration of 1×MICₘₒₙₒ or less, zidovudine may be used at a concentration of 1×MICₘₒₙₒ or less and rifampicin may be used at a concentration of 1×MICₘₒₙₒ or less. Preferably, ceftazidime may be used at a concentration of 1×MICₘₒₙₒ or less, zidovudine may be used at a concentration of 1×MICₘₒₙₒ or less and rifampicin may be used at a concentration of 0.125 to 1×MICₘₒₙₒ. More preferably, ceftazidime may be used at a concentration of 0.5×MICₘₒₙₒ or less, zidovudine may be used at a concentration of 1×MICₘₒₙₒ or less, and rifampicin is used at a concentration of 0.25 to 1×MICₘₒₙₒ.

In various embodiments the concentration of polymyxin E or polymyxin B may be 1×MICₘₒₙₒ or less for the bacteria against which the combination is being used. Preferably the concentration of polymyxin E or polymyxin B may be 0.5×MICₘₒₙₒ or less for the bacteria against which the combination is being used. More preferably the concentration of polymyxin E or polymyxin B may be 0.25×MICₘₒₙₒ or less for the bacteria against which the combination is being used. Most preferably the concentration of polymyxin E or polymyxin B may be 0.125×MICₘₒₙₒ or less for the bacteria against which the combination is being used.

As an example, the polymyxin E or polymyxin B concentration may be about 2 mg/L or less in a combination showing synergy against ESBL E. coli, preferably about 0.06 to about 2 mg/L.

As a further example, the polymyxin E or polymyxin B concentration may be about 4 mg/L or less in a combination showing synergy against K. pneumoniae CPE, preferably about 0.25 to about 4 mg/L.

As a further example, the polymyxin E or polymyxin B concentration may be about 512 mg/L or less in a combination showing synergy against P. aeruginosa, preferably about 0.03125 to about 512 mg/L, more preferably about 0.5 to about 512 mg/L, even more preferably about 0.5 to about 64 mg/L.

As a further example, the polymyxin E or polymyxin B concentration may be about 256 mg/L or less in a combination showing synergy against A. baumanii, preferably about 128 mg/L or less. In various embodiments, the polymyxin E or polymyxin B concentration may be about about 0.125 to about 256 mg/L, more preferably about 8 to about 256 mg/L. In various embodiments, the polymyxin E or polymyxin B concentration may be about about 0.125 to about 128 mg/L, more preferably about 8 to about 128 mg/L.

The concentration ranges for polymyxin E/B may be combined with those for each of ceftazidime and zidovudine above. Alternatively, the concentration ranges for polymyxin E/B may be combined with each of zidovudine/doxycycline/fosfomycin and levofloxacin/doxycycline/meropenem/gentamicin/rifampicin defined herein.

In various embodiments, ceftazidime is used at a concentration of about 0.5 to about 32 mg/L, zidovudine is used at a concentration of about 0.03 to about 1 mg/L and polymyxin E/B is used at a concentration of about 0.06 to about 2 mg/L in a combination showing synergy against ESBL E. coli. Preferably the ceftazidime concentration may be about 0.5 to about 16 mg/L, the zidovudine concentration may be about 0.03 to about 1 mg/L and the polymyxin E/B concentration may be about 0.5 to about 2 mg/L in a combination showing synergy against ESBL E. coli. Most preferably the ceftazidime concentration may be about 0.5 to about 4 mg/L, the zidovudine concentration may be about 0.03 to about 1 mg/L and the polymyxin E/B concentration may be about 0.5 to about 2 mg/L, most preferably about 1 mg/L to about 2 mg/L in a combination showing synergy against ESBL E. coli.

In various embodiments ceftazidime is used at a concentration of about 0.5 to about 4 mg/L, zidovudine is used at a concentration of about 0.125 to about 1 mg/L and polymyxin E/B is used at a concentration of about 0.5 to about 2 mg/L in a combination showing synergy against ESBL E. coli. Preferably, ceftazidime may be used at a concentration of about 0.5 to about 4 mg/L, zidovudine may be used at a concentration of about 0.5 to about 1 mg/L and polymyxin E/B is used at a concentration of about 0.06 to about 2 mg/L in a combination showing synergy against ESBL E. coli.

In various embodiments, ceftazidime is used at a concentration of about 0.015625 to about 512 mg/L, zidovudine is used at a concentration of about 0.5 to about 512 mg/L and polymyxin E/B is used at a concentration of about 0.03125 to about 512 mg/L in a combination showing synergy against P. aeruginosa. Preferably the ceftazidime concentration may be about 0.5 to about 512 mg/L, the zidovudine concentration may be about 2 to about 512 mg/L and the polymyxin E/B concentration may be about 0.5 to about 512 mg/L in a combination showing synergy against P. aeruginosa. Most preferably the ceftazidime concentration may be about 0.5 to about 4 mg/L, the zidovudine concentration may be about 8 to about 512 mg/L and the polymyxin E/B concentration may be about 0.5 to about 512 mg/L, most preferably about 0.5 mg/L to about 64 mg/L in a combination showing synergy against P. aeruginosa.

In various embodiments, ceftazidime is used at a concentration of about 32 to about 512 mg/L, zidovudine is used at a concentration of about 0.5 to about 512 mg/L and polymyxin E/B is used at a concentration of about 0.125 to about 256 mg/L in a combination showing synergy against A. baumanii. Preferably the ceftazidime concentration may be about 32 to about 512 mg/L, the zidovudine concentration may be about 0.5, 2, or 4 mg/L to about 512 mg/L and the polymyxin E/B concentration may be about 0.125 to about 256 mg/L, more preferably about 8 to about 256 mg/L, in a combination showing synergy against A. baumanii. More preferably, the ceftazidime concentration may be about 32 to about 512 mg/L, the zidovudine concentration may be about 0.5, 2, or 4 mg/L to about 128 mg/L and the polymyxin E/B concentration may be about 0.125 to about 256 mg/L, more preferably about 8 to about 256 mg/L, in a combination showing synergy against A. baumanii. Even more preferably, the ceftazidime concentration may be about 32 to about 128 mg/L, the zidovudine concentration may be about 0.5, 2, or 4 mg/L to about 128 mg/L and the polymyxin E/B concentration may be about 0.125 to about 256 mg/L, more preferably about 8 to about 256 mg/L, in a combination showing synergy against A. baumanii.

The concentrations of each of ceftazidime, zidovudine and polymyxin E may also be expressed as factors of MICₘₒₙₒ. For example, ceftazidime may be used at a concentration of 1×MICₘₒₙₒ or less, zidovudine may be used at a concentration of 0.5 to 1×MICₘₒₙₒ and polymyxin E may be used at a concentration of 1×MICₘₒₙₒ or less. Preferably, ceftazidime may be used at a concentration of 1×MICₘₒₙₒ or less, zidovudine may be used at a concentration of 0.5 to 1×MICₘₒₙₒ or less and polymyxin E may be used at a concentration of 0.125 to 1×MICₘₒₙₒ. Alternatively, ceftazidime may be used at a concentration of 0.5×MICₘₒₙₒ or less, zidovudine may be used at a concentration of 1×MICₘₒₙₒ or less, and polymyxin E is used at a concentration of 0.5 to 1×MICₘₒₙₒ.

In various embodiments the concentration of meropenem may be 1×MICₘₒₙₒ or less for the bacteria against which the combination is being used. Preferably the concentration of meropenem may be 0.5×MICₘₒₙₒ or less for the bacteria against which the combination is being used. More preferably the concentration of meropenem may be 0.25×MICₘₒₙₒ or less for the bacteria against which the combination is being used. Most preferably the concentration of meropenem is 0.125×MICₘₒₙₒ or less for the bacteria against which the combination is being used.

As an example, the meropenem concentration may be about 0.02 mg/L or less in a combination showing synergy against ESBL E. coli, preferably about 9×10⁻⁴ to about 0.02 mg/L.

As a further example, the meropenem concentration may be about 32 mg/L or less in a combination showing synergy against P. aeruginosa, preferably about 2 to about 32 mg/L, more preferably about 4 to about 32 mg/L.

As a further example, the meropenem concentration may be about 32 mg/L or less in a combination showing synergy against MRSA, preferably about 4 to about 32 mg/L.

The concentration ranges for meropenem may be combined with those for each of ceftazidime/polymyxin E/B and doxycycline/fosfomycin/zidovudine defined herein.

In various embodiments, ceftazidime is used at a concentration of about 0.015625 to about 512 mg/L, zidovudine is used at a concentration of about 0.5 to about 512 mg/L and meropenem is used at a concentration of about 2 to about 32 mg/L in a combination showing synergy against P. aeruginosa. Preferably the ceftazidime concentration may be about 0.015625 to about 512 mg/L, the zidovudine concentration may be about 2 to about 512 mg/L and the meropenem concentration may be about 2 to about 32 mg/L in a combination showing synergy against P. aeruginosa. Most preferably the ceftazidime concentration may be about 0.015625 to about 512 mg/L, the zidovudine concentration may be about 8 to about 512 mg/L and the meropenem concentration may be about 2 to about 32 mg/L, most preferably about 4 to about 32 mg/L, in a combination showing synergy against P. aeruginosa.

In various embodiments, ceftazidime is used at a concentration of about 0.5 to about 512 mg/L, zidovudine is used at a concentration of about 1 to about 512 mg/L and meropenem is used at a concentration of about 2 to about 32 mg/L in a combination showing synergy against P. aeruginosa. Preferably the ceftazidime concentration may be about 0.5 to about 512 mg/L, the zidovudine concentration may be about 2 to about 512 mg/L and the meropenem concentration may be about 2 to about 32 mg/L in a combination showing synergy against P. aeruginosa. More preferably the ceftazidime concentration may be about 0.5 to about 512 mg/L, the zidovudine concentration may be about 8 to about 512 mg/L and the meropenem concentration may be about 2 to about 32 mg/L, more preferably about 4 to about 32 mg/L, in a combination showing synergy against P. aeruginosa. Most preferably the ceftazidime concentration may be about 0.5 to about 4 mg/L, the zidovudine concentration may be about 8 to about 64 mg/L and the meropenem concentration may be about 4 to about 32 mg/L in a combination showing synergy against P. aeruginosa.

The concentrations of each of ceftazidime, zidovudine and meropenem may also be expressed as factors of MICₘₒₙₒ. For example, ceftazidime may be used at a concentration of 1×MICₘₒₙₒ or less, zidovudine may be used at a concentration of 1×MICₘₒₙₒ or less and meropenem may be used at a concentration of 1×MICₘₒₙₒ or less. Preferably, ceftazidime may be used at a concentration of 0.5×MICₘₒₙₒ or less, zidovudine may be used at a concentration of 1×MICₘₒₙₒ or less and meropenem may be used at a concentration of 0.25 to 1×MICₘₒₙₒ. More preferably, ceftazidime may be used at a concentration of 0.25×MICₘₒₙₒ or less, zidovudine may be used at a concentration of 1×MICₘₒₙₒ or less, and meropenem may be used at a concentration of 0.25 to 1×MICₘₒₙₒ or less. Most preferably, ceftazidime is used at a concentration of 0.125×MICₘₒₙₒ or less (e.g. 0.0625 MICₘₒₙₒ), zidovudine may be used at a concentration of 0.5 to 1×MICₘₒₙₒ, and meropenem may be used at a concentration of 0.25 to 1×MICₘₒₙₒ.

The lower limit for the MICₘₒₙₒ ranges defined herein is not limited. Where one is not specified, it is preferably 1/512^{th} MICₘₒₙₒ, 1/256^{th} MICₘₒₙₒ, 1/128^{th} MICₘₒₙₒ, 1/64^{th} MICₘₒₙₒ, 1/32^{nd} MICₘₒₙₒ, or 0.0625 MICₘₒₙₒ. For example, “0.5×MICₘₒₙₒ or less” becomes “0.5×MICₘₒₙₒ to 0.0625 MICₘₒₙₒ.

The concentration ranges of meropenem may be combined with those for each of levofloxacin and ceftazidime. In various embodiments, ceftazidime is used at a concentration of about 1 to about 32 mg/L, the concentration of levofloxacin is about 0.03125 to about 1 mg/L, and the concentration of meropenem is about 4 to about 32 mg/L in a combination showing synergy against MRSA. Preferably the ceftazidime concentration is about 1 to about 32 mg/L, the concentration of levofloxacin is about 0.125 to about 1 mg/L and the meropenem concentration is about 4 to about 32 mg/L in a combination showing synergy against MRSA.

The concentrations of each of ceftazidime, levofloxacin and meropenem may also be expressed as factors of MICₘₒₙₒ. For example, ceftazidime may be used at a concentration of 1×MICₘₒₙₒ or less, levofloxacin may be used at a concentration of 1×MICₘₒₙₒ or less and meropenem may be used at a concentration of 1×MICₘₒₙₒ or less. Preferably, ceftazidime may be used at a concentration of 0.5×MICₘₒₙₒ or less, levofloxacin may be used at a concentration of 1×MICₘₒₙₒ or less and meropenem may be used at a concentration of 0.25 to 1×MICₘₒₙₒ. More preferably, ceftazidime may be used at a concentration of 0.25×MICₘₒₙₒ or less, levofloxacin may be used at a concentration of 1×MICₘₒₙₒ or less, and meropenem may be used at a concentration of 0.25 to 1×MICₘₒₙₒ or less. Most preferably, ceftazidime is used at a concentration of 0.125×MICₘₒₙₒ or less (e.g. 0.0625 MICₘₒₙₒ), levofloxacin may be used at a concentration of 0.5 to 1×MICₘₒₙₒ, and meropenem may be used at a concentration of 0.25 to 1×MICₘₒₙₒ. In various embodiments, ceftazidime is used at a concentration of about 0.5 to about 32 mg/L, doxycycline is used at a concentration of about 0.0625 to about 1 mg/L and meropenem is used at a concentration of about 9×10⁻⁴ to about 0.02 mg/L in a combination showing synergy against ESBL E. coli. Preferably the ceftazidime concentration may be about 0.5 to about 16 mg/L, the doxycycline concentration may be about 0.0625 to about 1 mg/L and the meropenem concentration may be about 0.0156 mg/L in a combination showing synergy against ESBL E. coli. Most preferably the ceftazidime concentration may be about 0.5 to about 4 mg/L, the doxycycline concentration may be about 0.625 to about 1 mg/L and the meropenem concentration may be about 0.156 mg/L in a combination showing synergy against ESBL E. coli.

In various embodiments ceftazidime is used at a concentration of about 0.5 to about 4 mg/L, doxycycline is used at a concentration of about 0.25 to about 1 mg/L and meropenem is used at a concentration of about 3×10⁻³ to about 0.02 mg/L in a combination showing synergy against ESBL E. coli. Preferably, ceftazidime may be used at a concentration of about 0.5 to about 2 mg/L, doxycycline may be used at a concentration of about 0.25 to about 1 mg/L and meropenem is used at a concentration of about 3×10⁻³ to about 0.02 mg/L, e.g. 7×10⁻³ to about 0.02 mg/L in a combination showing synergy against ESBL E. coli.

The concentrations of each of ceftazidime, doxycycline and meropenem may also be expressed as factors of MICₘₒₙₒ. For example, ceftazidime may be used at a concentration of 1×MICₘₒₙₒ or less, doxycycline may be used at a concentration of 1×MICₘₒₙₒ or less and meropenem may be used at a concentration of 1×MICₘₒₙₒ or less. Preferably, ceftazidime may be used at a concentration of 0.5×MICₘₒₙₒ or less, doxycycline may be used at a concentration of 1×MICₘₒₙₒ or less and meropenem may be used at a concentration of 0.25 to 1×MICₘₒₙₒ. More preferably, ceftazidime may be used at a concentration of 0.25×MICₘₒₙₒ or less, doxycycline may be used at a concentration of 1×MICₘₒₙₒ or less, and meropenem may be used at a concentration of 0.25 to 1×MICₘₒₙₒ or less. Most preferably, ceftazidime is used at a concentration of 0.125×MICₘₒₙₒ or less (e.g. 0.0625 MICₘₒₙₒ), doxycycline may be used at a concentration of 0.5 to 1×MICₘₒₙₒ, and meropenem may be used at a concentration of 0.25 to 1×MICₘₒₙₒ.

The lower limit for the MICₘₒₙₒ ranges defined herein is not limited. Where one is not specified, it is preferably 1/512^{th} MICₘₒₙₒ, 1/256^{th} MICₘₒₙₒ, 1/128^{th} MICₘₒₙₒ, 1/64^{th} MICₘₒₙₒ, 1/32^{nd} MICₘₒₙₒ, or 0.0625 MICₘₒₙₒ. For example, “0.5×MICₘₒₙₒ or less” becomes “0.5×MICₘₒₙₒ to 0.0625 MICₘₒₙₒ.

In various embodiments the concentration of fosfomycin may be 1×MICₘₒₙₒ or less for the bacteria against which the combination is being used. Preferably the concentration of fosfomycin may be 0.5×MICₘₒₙₒ or less for the bacteria against which the combination is being used. More preferably the concentration of fosfomycin may be 0.25×MICₘₒₙₒ or less for the bacteria against which the combination is being used. Most preferably, the concentration of fosfomycin is 0.125×MICₘₒₙₒ or less for the bacteria against which the combination is being used.

As an example, the fosfomycin concentration may be about 64 mg/L or less in a combination showing synergy against K. pneumoniae CPE, preferably about 4 to about 64 mg/L.

As a further example, the fosfomycin concentration may be about 256 mg/L or less in a combination showing synergy against P. aeruginosa, preferably about 128 mg/L or less. Preferably, the fosfomycin concentration may be about 4 to about 256 mg/L, more preferably about 16 to about 128 mg/L in a combination showing synergy against P. aeruginosa.

As a further example, the fosfomycin concentration may be about 256 mg/L or less in a combination showing synergy against A. baumanii, preferably about 32 to about 256 mg/L.

As a further example, the fosfomycin concentration may be about 16 mg/L or less in a combination showing synergy against MRSA, preferably about 2 to about 16 mg/L.

The concentration ranges for fosfomycin may be combined with those for each of ceftazidime/polymyxin E/B, zidovudine/doxycycline and/or levofloxacin/doxycycline/meropenem/rifampicin/gentamicin/polymycin E/B as defined herein.

In various embodiments, ceftazidime is used at a concentration of about 0.25 to about 32 mg/L, doxycycline is used at a concentration of about 0.0625 to about 0.5 mg/L and fosfomycin is used at a concentration of about 4 or about 16 to about 64 mg/L in a combination showing synergy against K. pneumoniae CPE. Preferably the ceftazidime concentration may be about 0.25 to about 16 mg/L, the doxycycline concentration may be about 0.125 to about 0.5 mg/L and the fosfomycin concentration may be about 4 or about 16 to 64 mg/L in a combination showing synergy against K. pneumoniae CPE. Most preferably the ceftazidime concentration may be about 0.5 to about 4 mg/L, the doxycycline concentration may be about 0.5 mg/L and the fosfomycin concentration may be about 4 to about 64 mg/L in a combination showing synergy against K. pneumoniae CPE.

The concentrations of each of ceftazidime, doxycycline and fosfomycin may also be expressed as factors of MICₘₒₙₒ. For example, ceftazidime may be used at a concentration of 1×MICₘₒₙₒ or less, doxycycline may be used at a concentration of 1×MICₘₒₙₒ or less and fosfomycin may be used at a concentration of 1×MICₘₒₙₒ or less. Preferably, ceftazidime may be used at a concentration of 0.5×MICₘₒₙₒ or less, doxycycline may be used at a concentration of 1×MICₘₒₙₒ or less and fosfomycin may be used at a concentration of 0.25 to 1×MICₘₒₙₒ. More preferably, ceftazidime may be used at a concentration of 0.25×MICₘₒₙₒ or less, doxycycline may be used at a concentration of 1×MICₘₒₙₒ or less, and fosfomycin may be used at a concentration of 0.25 to 1×MICₘₒₙₒ or less. Most preferably, ceftazidime is used at a concentration of 0.125×MICₘₒₙₒ, doxycycline may be used at a concentration of 0.125 to 1×MICₘₒₙₒ or less, and fosfomycin may be used at a concentration of 0.25 to 1×MICₘₒₙₒ.

In various embodiments, ceftazidime is used at a concentration of about 0.25 to about 32 mg/L, fosfomycin is used at a concentration of about 4 to about 64 mg/L, and rifampicin is used at a concentration of about 1 to 8 mg/L in a combination showing synergy against K. pneumoniae CPE. Preferably the ceftazidime concentration may be about 0.25 to about 16 mg/L, fosfomycin concentration may be about 4 to 64 mg/L and the rifampicin concentration may be about 1 to 8 mg/L in a combination showing synergy against K. pneumoniae CPE. Most preferably the ceftazidime concentration may be about 0.5 to about 4 mg/L, the fosfomycin concentration may be about 4 to about 64 mg/L and the rifampicin concentration may be about 1 to 8 mg/L in a combination showing synergy against K. pneumoniae CPE.

In various embodiments, ceftazidime is used at a concentration of about 0.5 to about 4 mg/L, the fosfomycin concentration may be about 4 to about 64 mg/L and the rifampicin concentration may be about 2 to 8 mg/L in a combination showing synergy against K. pneumoniae CPE. More preferably, ceftazidime is used at a concentration of about 0.5 to about 4 mg/L, the fosfomycin concentration may be about 8 to about 64 mg/L and the rifampicin concentration may be about 2 to 8 mg/L in a combination showing synergy against K. pneumoniae CPE.

The concentrations of each of ceftazidime, fosfomycin and rifampicin may also be expressed as factors of MICₘₒₙₒ. For example, ceftazidime may be used at a concentration of 1×MICₘₒₙₒ or less, fosfomycin may be used at a concentration of 1×MICₘₒₙₒ or less, and rifampicin may be used at a concentration of 1×MICₘₒₙₒ or less. Preferably, ceftazidime may be used at a concentration of 0.5×MICₘₒₙₒ or less, fosfomycin may be used at a concentration of 0.125 to 1×MICₘₒₙₒ, and rifampicin may be used at a concentration of 1×MICₘₒₙₒ or less. More preferably, ceftazidime may be used at a concentration of 0.25×MICₘₒₙₒ or less, fosfomycin may be used at a concentration of 0.25 to 1×MICₘₒₙₒ or less, and rifampicin may be used at a concentration of 1×MICₘₒₙₒ or less. Most preferably, ceftazidime is used at a concentration of 0.125×MICₘₒₙₒ, fosfomycin may be used at a concentration of 0.5 to 1×MICₘₒₙₒ (e.g. 0.5 MICₘₒₙₒ) and rifampicin may be used at a concentration of 1×MICₘₒₙₒ or less.

As noted above, the concentration ranges for fosfomycin may be combined with those for each of ceftazidime and levofloxacin.

In various embodiments, ceftazidime is used at a concentration of about 1 to about 32 mg/L, fosfomycin is used at a concentration of about 2 to about 16 mg/L, and levofloxacin is used at a concentration of about 0.0625 to about 1 mg/L in a combination showing synergy against MRSA. Preferably the ceftazidime concentration may be about 1 to about 32 mg/L, the fosfomycin concentration may be about 2 to about 16 mg/L and the levofloxacin concentration may be about 0.125 to about 1 mg/L in a combination showing synergy against MRSA.

The concentrations of each of ceftazidime, fosfomycin and levofloxacin may also be expressed as factors of MICₘₒₙₒ. For example, ceftazidime may be used at a concentration of 1×MICₘₒₙₒ or less, fosfomycin may be used at a concentration of 1×MICₘₒₙₒ or less, and levofloxacin may be used at a concentration of 1×MICₘₒₙₒ or less. Preferably, ceftazidime may be used at a concentration of 0.5×MICₘₒₙₒ or less, fosfomycin may be used at a concentration of 0.125 to 1×MICₘₒₙₒ, and levofloxacin may be used at a concentration of 1×MICₘₒₙₒ or less. More preferably, ceftazidime may be used at a concentration of 0.25×MICₘₒₙₒ or less, fosfomycin may be used at a concentration of 0.25 to 1×MICₘₒₙₒ or less, and levofloxacin may be used at a concentration of 1×MICₘₒₙₒ or less. Most preferably, ceftazidime is used at a concentration of 0.125×MICₘₒₙₒ, fosfomycin may be used at a concentration of 0.5 to 1×MICₘₒₙₒ (e.g. 0.5 MICₘₒₙₒ) and levofloxacin may be used at a concentration of 1×MICₘₒₙₒ or less.

In various embodiments, polymyxin E/B is used at a concentration of about 0.03125 to about 512 mg/L, zidovudine is used at a concentration of about 0.03125 to about 512 mg/L and fosfomycin is used at a concentration of about 4 to about 256 mg/L in a combination showing synergy against P. aeruginosa. Preferably the polymyxin E/B concentration may be about 0.03125 to about 512 mg/L, the zidovudine concentration may be about 0.5 to about 512 mg/L and the fosfomycin concentration may be about 4 to about 256 mg/L in a combination showing synergy against P. aeruginosa. More preferably the polymyxin E/B concentration may be about 0.5 to about 512 mg/L, the zidovudine concentration may be about 0.5 to about 512 mg/L and the fosfomycin concentration may be about 16 to about 128 mg/L in a combination showing synergy against P. aeruginosa. Most preferably the polymyxin E/B concentration may be about 0.5 to about 1 mg/L, the zidovudine concentration may be about 0.5 to about 1 mg/L and the fosfomycin concentration may be about 16 to about 128 mg/L in a combination showing synergy against P, aeruginosa.

In various embodiments, polymyxin E/B is used at a concentration of about 0.06 to about 128 mg/L, zidovudine is used at a concentration of about 0.03125 to about 512 mg/L and fosfomycin is used at a concentration of about 32 to about 256 mg/L in a combination showing synergy against A. baumanii. Preferably the polymyxin E/B concentration may be about 0.5 to about 128 mg/L, the zidovudine concentration may be about 0.5 to about 512 mg/L and the fosfomycin concentration may be about 32 to about 256 mg/L in a combination showing synergy against A. baumanii. More preferably, the polymyxin E/B concentration may be about 0.5 to about 2 mg/L, the zidovudine concentration may be about 0.5 to about 1 mg/L, and the fosfomycin concentration may be about 32 to about 256 mg/L in a combination showing synergy against A. baumanii.

The concentrations of each of polymyxin E/B, zidovudine and fosfomycin may also be expressed as factors of MICₘₒₙₒ. For example, polymyxin E/B may be used at a concentration of 1×MICₘₒₙₒ or less, zidovudine may be used at a concentration of 1×MICₘₒₙₒ or less, and fosfomycin may be used at a concentration of 1×MICₘₒₙₒ or less. Preferably, polymyxin E/B may be used at a concentration of 0.5×MICₘₒₙₒ or less, zidovudine may be used at a concentration of 0.125 to 1×MICₘₒₙₒ, and fosfomycin may be used at a concentration of 1×MICₘₒₙₒ or less. More preferably, polymyxin E/B may be used at a concentration of 0.25×MICₘₒₙₒ or less, zidovudine may be used at a concentration of 0.25 to 1×MICₘₒₙₒ or less, and fosfomycin may be used at a concentration of 1×MICₘₒₙₒ or less. Most preferably, polymyxin E/B is used at a concentration of 0.125×MICₘₒₙₒ, zidovudine may be used at a concentration of 0.5 to 1×MICₘₒₙₒ (e.g. 0.5 MICₘₒₙₒ) and fosfomycin may be used at a concentration of 1×MICₘₒₙₒ or less.

In various embodiments, colistin/polymyxin E is used at a concentration of about 0.125 to about 4 mg/L, doxycycline is used at a concentration of about 0.0625 to about 1 mg/L, and rifampicin is used at a concentration of about 0.25 to 4 mg/L in a combination showing synergy against E. coli ESBL. Preferably the colistin concentration may be about 0.125 to about 2 mg/L, the doxycycline concentration may be about 0.0625 to about 1 mg/L and the rifampicin concentration may be about 0.25 to 4 mg/L in a combination showing synergy against E. coli ESBL. Most preferably the colistin concentration may be about 0.25 to about 0.5 mg/L, the doxycline concentration may be about 0.0625 to about 1 mg/L and the rifampicin concentration may be about 0.25 to 4 mg/L in a combination showing synergy against E. coli ESBL.

The concentrations of each of colistin, doxycycline and rifampicin may also be expressed as factors of MICₘₒₙₒ. For example, colistin may be used at a concentration of 1×MICₘₒₙₒ or less, doxycycline may be used at a concentration of 1×MICₘₒₙₒ or less, and rifampicin may be used at a concentration of 1×MICₘₒₙₒ or less. Preferably, colistin may be used at a concentration of 0.5×MICₘₒₙₒ or less, doxycycline may be used at a concentration of 1×MICₘₒₙₒ or less, and rifampicin may be used at a concentration of 1×MICₘₒₙₒ or less. More preferably, colistin may be used at a concentration of 0.125×MICₘₒₙₒ or less, doxycline may be used at a concentration of 0.125 to 1×MICₘₒₙₒ or less, and rifampicin may be used at a concentration of 1×MICₘₒₙₒ or less. Most preferably, colistin is used at a concentration of 0.125×MICₘₒₙₒ, doxycycline may be used at a concentration of 0.125 to 1×MICₘₒₙₒ and rifampicin may be used at a concentration of 1×MICₘₒₙₒ or less.

In various embodiments, colistin/polymyxin E is used at a concentration of about 0.125 to about 4 mg/L, fosfomycin is used at a concentration of about 4 to 64 mg/L, and levofloxacin is used at a concentration of about 0.1565 to 0.25 mg/L in a combination showing synergy against E. coli ESBL. Preferably the colistin concentration may be about 0.125 to about 2 mg/L, the fosfomycin concentration may be about 4 or about 16 to 64 mg/L and the levofloxacin concentration may be about 0.0156 to 0.25 mg/L in a combination showing synergy against E. coli ESBL. Most preferably the colistin concentration may be about 0.5 mg/L, the fosfomycin concentration may be about 4 or 16 to 64 mg/L and the levofloxacin concentration may be about 0.03 or 0.125 to 0.25 mg/L in a combination showing synergy against E. coli ESBL.

The concentrations of each of colistin, fosfomycin and levofloxacin may also be expressed as factors of MICₘₒₙₒ. For example, colistin may be used at a concentration of 1×MICₘₒₙₒ or less, fosfomycin may be used at a concentration of 1×MICₘₒₙₒ or less, and levofloxacin may be used at a concentration of 1×MICₘₒₙₒ or less. Preferably, colistin may be used at a concentration of 0.5×MICₘₒₙₒ or less, fosfomycin may be used at a concentration of 0.5 to 1×MICₘₒₙₒ, and levofloxacin may be used at a concentration of 0.625 to 0.5×MICₘₒₙₒ. Most preferably, colistin is used at a concentration of 0.125×MICₘₒₙₒ, fosfomycin may be used at a concentration of 0.5 to 1×MICₘₒₙₒ and levofloxacin may be used at a concentration of 1×MICₘₒₙₒ or less, e.g. 0.0625 to 0.5×MICₘₒₙₒ.

In various embodiments the concentration of gentamicin may be 1×MICₘₒₙₒ or less for the bacteria against which the combination is being used. Preferably the concentration of gentamicin may be 0.5×MICₘₒₙₒ or less for the bacteria against which the combination is being used. More preferably the concentration of gentamicin may be 0.25×MICₘₒₙₒ or less for the bacteria against which the combination is being used. Most preferably, the concentration of gentamicin is 0.125×MICₘₒₙₒ or less for the bacteria against which the combination is being used.

As an example, the gentamicin concentration may be about 2 mg/L or less in a combination showing synergy against K. pneumoniae CPE, preferably about 0.125 to about 2 mg/L, more preferably about 0.25 to about 2 mg/L.

In various embodiments, colistin/polymyxin E is used at a concentration of about 0.125 to about 4 mg/L, fosfomycin is used at a concentration of about 4 to 64 mg/L, and gentamicin is used at a concentration of about 0.125 to 2 mg/L in a combination showing synergy against K. pneumoniae CPE. Preferably the colistin concentration may be about 0.125 to about 2 mg/L, the fosfomycin concentration may be about 4 or about 16 to 64 mg/L and the gentamicin concentration may be about 0.125 to 2 mg/L in a combination showing synergy against K. pneumoniae CPE. Most preferably the colistin concentration may be about 0.5 mg/L, the fosfomycin concentration may be about 4 or 16 to 64 mg/L and the gentamicin concentration may be about 0.125 to 2 mg/L in a combination showing synergy against K. pneumoniae CPE.

The concentrations of each of colistin, fosfomycin and gentamicin may also be expressed as factors of MICₘₒₙₒ. For example, colistin may be used at a concentration of 1×MICₘₒₙₒ or less, fosfomycin may be used at a concentration of 1×MICₘₒₙₒ or less, and gentamicin may be used at a concentration of 1×MICₘₒₙₒ or less. Preferably, colistin may be used at a concentration of 0.5×MICₘₒₙₒ or less, fosfomycin may be used at a concentration of 0.0625 or 0.5 to 1×MICₘₒₙₒ, and gentamicin may be used at a concentration of 0.625 to 0.5×MICₘₒₙₒ. More preferably, colistin may be used at a concentration of 0.125×MICₘₒₙₒ or less, fosfomycin may be used at a concentration of 0.5 to 1×MICₘₒₙₒ, and gentamicin may be used at a concentration of 1×MICₘₒₙₒ or less. Most preferably, colistin is used at a concentration of 0.125×MICₘₒₙₒ, fosfomycin may be used at a concentration of 0.5 to 1×MICₘₒₙₒ and gentamicin may be used at a concentration of 1×MICₘₒₙₒ or less, e.g. 0.0625 to 0.5×MICₘₒₙₒ.

The concentration ranges for meropenem may also be combined with those for each of ceftazidime/fosfomycin, zidovudine, and polymyxin E/colistin; e.g. in a combination comprising four antimicrobial agents showing synergy against P. aeruginosa.

In various embodiments, zidovudine is used at a concentration of about 1 to about 512 mg/L, colistin is used at a concentration of about 2 to about 512 mg/L, ceftazidime is used at a concentration of about 1 to about 512 mg/L, and meropenem is used at a concentration of about 1 to about 32 mg/L in a combination showing synergy against P. aeruginosa. Preferably, the concentration of zidovudine may be about 1 to about 64 mg/L, the concentration of colistin is about 2 to about 512 mg/L, the concentration of ceftazidime is about 1 to 512 mg/L and the concentration of meropenem is about 1 to about 32 mg/L in a combination showing synergy against P. aeruginosa. More preferably, the concentration of zidovudine may be about 1 to about 64 mg/L, the concentration of colistin is about 2 to about 512 mg/L, the concentration of ceftazidime is about 1 to 512 mg/L and the concentration of meropenem is about 1 to about 4 mg/L in a combination showing synergy against P. aeruginosa.

The concentrations of each of zidovudine, colistin, ceftazidime and meropenem may also be expressed as factors of MICₘₒₙₒ. For example, zidovudine may be used at a concentration of 1×MICₘₒₙₒ or less, colistin may be used at a concentration of 1×MICₘₒₙₒ or less, ceftazidime may be used at a concentration of 1×MICₘₒₙₒ or less, and meropenem may be used at a concentration of 1×MICₘₒₙₒ or less. Preferably, zidovudine may be used at a concentration of 1×MICₘₒₙₒ or less, colistin may be used at a concentration of 1×MICₘₒₙₒ or less, ceftazidime may be used at a concentration of 1×MICₘₒₙₒ, and meropenem may be used at a concentration of 1×MICₘₒₙₒ or less. Alternatively, zidovudine may be used at a concentration of 0.5 to 1×MICₘₒₙₒ, colistin may be used at a concentration of 1×MICₘₒₙₒ, ceftazidime is used at a concentration of 1×MICₘₒₙₒ or less, and meropenem may be used at a concentration of 1×MICₘₒₙₒ or less. Alternatively, zidovudine may be used at a concentration of 1×MICₘₒₙₒ, colistin may be used at a concentration of 0.5 to 1×MICₘₒₙₒ, ceftazidime is used at a concentration of 1×MICₘₒₙₒ or less, and meropenem may be used at a concentration of 1×MICₘₒₙₒ or less. Alternatively, zidovudine may be used at a concentration of 1×MICₘₒₙₒ, colistin may be used at a concentration of 1×MICₘₒₙₒ, ceftazidime is used at a concentration of 0.5 to 1×MICₘₒₙₒ or less, and meropenem may be used at a concentration of 1×MICₘₒₙₒ or less. Alternatively, zidovudine may be used at a concentration of 1×MICₘₒₙₒ, colistin may be used at a concentration of 1×MICₘₒₙₒ, ceftazidime is used at a concentration of 1×MICₘₒₙₒ or less, and meropenem may be used at a concentration of 0.5 to 1×MICₘₒₙₒ or less.

The lower limit for the MICₘₒₙₒ ranges defined herein is not limited. Where one is not specified, it is preferably 1/512^{th} MICₘₒₙₒ, 1/256^{th} MICₘₒₙₒ, 1/128^{th} MICₘₒₙₒ, 1/64^{th} MICₘₒₙₒ, 1/32^{nd} MICₘₒₙₒ, or 0.0625 MICₘₒₙₒ. For example, “0.5×MICₘₒₙₒ or less” becomes “0.5×MICₘₒₙₒ to 0.0625×MICₘₒₙₒ.

In various embodiments, zidovudine is used at a concentration of about 1 to about 512 mg/L, colistin is used at a concentration of about 16 to about 512 mg/L, fosfomycin is used at a concentration of about 4 to about 256 mg/L, and meropenem is used at a concentration of about 1 to about 32 mg/L in a combination showing synergy against P. aeruginosa. Preferably, zidovudine is used at a concentration of about 1 to about 512 mg/L, colistin is used at a concentration of about 32 to about 512 mg/L, fosfomycin is used at a concentration of about 4 to about 256 mg/L, and meropenem is used at a concentration of about 1 to about 32 mg/L in a combination showing synergy against P. aeruginosa. More preferably, zidovudine is used at a concentration of about 1 to about 512 mg/L, colistin is used at a concentration of about 32 to about 64 mg/L, fosfomycin is used at a concentration of about 4 to about 256 mg/L, and meropenem is used at a concentration of about 1 to about 32 mg/L in a combination showing synergy against P. aeruginosa. Even more preferably, zidovudine is used at a concentration of about 1 to about 64 mg/L, colistin is used at a concentration of about 32 to about 64 mg/L, fosfomycin is used at a concentration of about 4 to about 128 mg/L, and meropenem is used at a concentration of about 1 to about 32 mg/L, more preferably 1 to about 4 mg/L, in a combination showing synergy against P. aeruginosa.

The concentrations of each of zidovudine, colistin, fosfomycin and meropenem may also be expressed as factors of MICₘₒₙₒ. For example, zidovudine may be used at a concentration of 1×MICₘₒₙₒ or less, colistin may be used at a concentration of 1×MICₘₒₙₒ or less, fosfomycin may be used at a concentration of 1×MICₘₒₙₒ or less, and meropenem may be used at a concentration of 1×MICₘₒₙₒ or less. Preferably, zidovudine may be used at a concentration of 1×MICₘₒₙₒ or less, colistin may be used at a concentration of 1×MICₘₒₙₒ or less, fosfomycin may be used at a concentration of 1×MICₘₒₙₒ, and meropenem may be used at a concentration of 1×MICₘₒₙₒ or less. Alternatively, zidovudine may be used at a concentration of 0.5 to 1×MICₘₒₙₒ, colistin may be used at a concentration of 1×MICₘₒₙₒ, fosfomycin is used at a concentration of 1×MICₘₒₙₒ or less, and meropenem may be used at a concentration of 1×MICₘₒₙₒ or less. Alternatively, zidovudine may be used at a concentration of 1×MICₘₒₙₒ, colistin may be used at a concentration of 0.5 to 1×MICₘₒₙₒ, fosfomycin is used at a concentration of 1×MICₘₒₙₒ or less, and meropenem may be used at a concentration of 1×MICₘₒₙₒ or less. Alternatively, zidovudine may be used at a concentration of 1×MICₘₒₙₒ, colistin may be used at a concentration of 1×MICₘₒₙₒ, fosfomycin is used at a concentration of 0.5 to 1×MICₘₒₙₒ or less, and meropenem may be used at a concentration of 1×MICₘₒₙₒ or less. Alternatively, zidovudine may be used at a concentration of 1×MICₘₒₙₒ, colistin may be used at a concentration of 1×MICₘₒₙₒ, fosfomycin is used at a concentration of 1×MICₘₒₙₒ or less, and meropenem may be used at a concentration of 0.5 to 1×MICₘₒₙₒ or less.

Formulations suitable for oral administration may be presented as discrete units such as capsules, cachets or tablets (e.g. chewable tablets in particular for paediatric administration), each containing a predetermined amount of active ingredient; as powder or granules; as a solution or suspension in an aqueous liquid or non-aqueous liquid; or as an oil-in-water liquid emulsion or water-in-oil liquid emulsion. The active ingredients may also be presented a bolus, electuary or paste.

A tablet may be made by compression or molding, optionally with one or more excipients. Compressed tablets may be prepared by compressing in a suitable machine the active ingredient in a free-flowing form such as a powder or granules, optionally mixed with other conventional excipients such as binding agents (e.g. syrup, acacia, gelatin, sorbitol, tragacanth, mucilage of starch, polyvinylpyrrolidone and/or hydroxymethyl cellulose), fillers (e.g. lactose, sugar, microcrystalline cellulose, maize-starch, calcium phosphate and/or sorbitol), lubricants (e.g. magnesium stearate, stearic acid, talc, polyethylene glycol and/or silica), disintegrants (e.g. potato starch, croscarmellose sodium and/or sodium starch glycolate) and wetting agents (e.g. sodium lauryl sulphate). Molded tablets may be made by molding in a suitable machine a mixture of the powdered active ingredient with an inert liquid diluent. The tablets may be optionally coated or scored and may be formulated so as to provide controlled release (e.g. delayed, sustained, or pulsed release, or a combination of immediate release and controlled release) of the active ingredients.

Alternatively, the active ingredients may be incorporated into oral liquid preparations such as aqueous or oily suspensions, solutions, emulsions, syrups or elixirs. Formulations containing the active ingredients may also be presented as a dry product for constitution with water or another suitable vehicle before use.

Such liquid preparations may contain conventional additives such as suspending agents (e.g. sorbitol syrup, methyl cellulose, glucose/sugar syrup, gelatin, hydroxymethyl cellulose, carboxymethyl cellulose, aluminium stearate gel and/or hydrogenated edible fats), emulsifying agents (e.g. lecithin, sorbitan mono-oleate and/or acacia), non-aqueous vehicles (e.g. edible oils, such as almond oil, fractionated coconut oil, oily esters, propylene glycol and/or ethyl alcohol), and preservatives (e.g. methyl or propyl p-hydroxybenzoates and/or sorbic acid).

Combinations for use according to the invention may be presented in a pack or dispenser device which may contain one or more unit dosage forms containing the active ingredients. The pack may, e.g. comprise metal or plastic foil, such as a blister pack. Where the compositions are intended for administration as three separate compositions these may be presented in the form of a twin pack.

Pharmaceutical compositions may also be prescribed to the patient in “patient packs” containing the whole course of treatment in a single package, usually a blister pack. Patient packs have an advantage over traditional prescriptions, where a pharmacist divides a patients' supply of a pharmaceutical from a bulk supply, in that the patient always has access to the package insert contained in the patient pack, normally missing in traditional prescriptions. The inclusion of the package insert has been shown to improve patient compliance with the physician's instructions.

The administration of the combination of the invention by means of a single patient pack, or patients packs of each composition, including a package insert directing the patient to the correct use of the invention is a desirable feature of this invention.

According to a further embodiment of the present invention there is provided a patient pack comprising at least one active of the combination according to the invention and an information insert containing directions on the use of the combination of the invention. In another embodiment of the invention, there is provided a double pack comprising in association for separate administration, an antimicrobial agent, preferably having biological activity against clinically latent microorganisms, and one or more of the compounds disclosed herein preferably having biological activity against clinically latent microorganisms.

The amount of active ingredients required for use in treatment will vary with the nature of the condition being treated and the age and condition of the patient, and will ultimately be at the discretion of the attendant physician. In general however, doses employed for adult human treatment will typically be in the range of 0.02 to 5000 mg per day, preferably 1 to 1500 mg per day. The desired dose may conveniently be presented in a single dose or as divided doses administered at appropriate intervals, e.g. as two, three or more sub-doses per day.

This information would therefore be readily obtained and understood by the person skilled in the art.

### Biological Tests

Test procedures that may be employed to determine the biological (e.g. bactericidal or antimicrobial) activity of the active ingredients include those known to persons skilled in the art for determining:
(a) bactericidal activity against clinically latent bacteria; and
(b) antimicrobial activity against log phase bacteria.

In relation to (a) above, methods for determining activity against clinically latent bacteria include a determination, under conditions known to those skilled in the art (such as those described in Nature Reviews, Drug Discovery 1, 895-910 (2002), the disclosures of which are hereby incorporated by reference), of Minimum Stationary-cidal Concentration (“MSC”) or Minimum Dormicidal Concentration (“MDC”) for a test compound.

By way of example, WO2000028074 describes a suitable method of screening compounds to determine their ability to kill clinically latent microorganisms. A typical method may include the following steps:
(1) growing a bacterial culture to stationary phase;
(2) treating the stationery phase culture with one or more antimicrobial agents at a concentration and or time sufficient to kill growing bacteria, thereby selecting a phenotypically resistant sub-population;
(3) incubating a sample of the phenotypically resistant subpopulation with one or more test compounds or agents; and
(4) assessing any antimicrobial effects against the phenotypically resistant subpopulation.

According to this method, the phenotypically resistant sub-population may be seen as representative of clinically latent bacteria which remain metabolically active in vivo and which can result in relapse or onset of disease.

In relation to (b) above, methods for determining activity against log phase bacteria include a determination, under standard conditions (i.e. conditions known to those skilled in the art, such as those described in WO 2005014585, the disclosures of which document are hereby incorporated by reference), of Minimum Inhibitory Concentration (“MIC”) or Minimum Bactericidal Concentration (“MBC”) for a test compound. Specific examples of such methods are described below.

### EXAMPLES

The antimicrobial agents were sourced from commercially available sources. They were prepared by being weighed and dissolved in water, PBS, DMSO or acidified water to a final concentration from 1-10 mg/mL. Antimicrobial solutions were diluted to 10× highest concentration used in the experiment and subsequently diluted 2× over a series not exceeding 11 serial dilutions. These left the operator with a maximum of 12 different, descending concentrations of the chosen antimicrobials.

The bacteria were obtained from Ninewells Hospital and Medical School in Dundee, Scotland. They were obtained as patient strains and characterised by Vitek 2 screening. To prepare for the Examples below, the bacteria were grown overnight in Mueller Hinton Cation adjusted broth or until confluent in media with or without supplements. The bacteria were measured for OD₆₀₀ and readings of <0.25 were returned to the incubator. Bacterial cultures were diluted in media until OD₆₀₀ reading register=<0.01, this indicated that the culture was at approximately 10⁶ CFU/ml.

All data herein was produced using the same chequerboard assay.

20 μL of antibiotic A (backbone), at a single dilution, was pipetted into all wells of a 96-well plate to be used.

20 μL of the lowest concentration of antibiotic B (first variable) was pipetted into column 1 of the 96-well plate. The second concentration (2-fold more concentrate) was pipetted into column 2. This process was repeated until all concentrations of antibiotic B to be used were complete.

20 μL of the lowest concentration of antibiotic C (second variable) were pipetted into row A of the 96-well plate. The second concentration (2-fold more concentrate) was pipetted into row B. This process was repeated until all concentrations of antibiotic C to be used were complete.

120 μL of sterile media (BHI/MHB2) was added.

20 ul of prepared bacterial culture was added.

Plates were sealed with lids and left to incubate for 16 hours, overnight.

Plates were read at OD₆₀₀ in a 96-well plate reader. These are the values reported in the chequerboards below.

As a tri-mer combination, two antibiotic concentrations were varied while one remained constant, the latter is the “backbone”. For the 4-mer combination, two antibiotic concentrations were varied while two remained constant, the latter is the “backbone”. This method is explained above along with the calculation of the FICI and the indicative value for synergy, indifference and antagonism.

The backbone for Comparative Examples 1 and 2 and Examples 3 to 5 was ceftazidime at 4 mg/L. The isolate for these Examples was resistant to cephalosporins and to ceftazidime at 32 mg/L. Hence, the 4 mg/L concentration of ceftazidime represented ⅛^{th} of the MIC for this isolate. This falls within the dosage guidance recommended by EUCAST (the European Committee on Antimicrobial Susceptibility Testing). EUCAST prepare Breakpoint tables for interpretation of MICs and zone diameters, see e.g. version 12.

### Comparative Example 1: Indifference Between Ceftazidime, Fosfomycin and Zidovudine

The triple combination of ceftazidime, fosfomycin and zidovudine was tested in the assay outlined above. Ceftazidime at 4 mg/L was the backbone and the combination was tested against ESBL E. coli. The highest concentration in each experiment was equal to 1×MIC for the isolate tested but this failed to inhibit growth. The following concentrations of zidovudine (AZT—x axis) and fosfomycin (fosfo—y axis) were used.

| | | AZT | | | | | | |
|---|---|---|---|---|---|---|---|---|
| | Mg/L | 1 | 0.5 | 0.25 | 0.125 | 0.0625 | 0.03125 | MIC |
| Fosfo | 1 | 0.092333 | 0.090333 | 0.269333 | 0.405333 | 0.388333 | 0.367333 | 1 |
| | 0.5 | 0.099333 | 0.012333 | 0.317333 | 0.469333 | 0.385333 | 0.431333 | half |
| | 0.25 | 0.005333 | 0.020333 | 0.306333 | 0.428333 | 0.406333 | 0.430333 | quarter |
| | 0.125 | 0.008333 | 0.012333 | 0.323333 | 0.419333 | 0.391333 | 0.439333 | 8th |
| | 0.0625 | 0.007333 | 0.051333 | 0.307333 | 0.397333 | 0.425333 | 0.434333 | 16th |
| | 0.03125 | 0.055333 | 0.084333 | 0.285333 | 0.418333 | 0.421333 | 0.453333 | 32nd |
| | MIC | 1 | half | quarter | 8th | 16th | 32nd | |

The results reported in the above table are OD600 values determined as described herein.

The ΣFIC was calculated in the same manner as shown below for Example 3. The ΣFIC=3 and FIC=2 for Comparative Example 1. Hence, the combination of ceftazidime, fosfomycin and zidovudine is indifferent or antagonistic vs ESBL E. coli.

Bold values in the above table (and similarly for the corresponding tables of the subsequent Examples) indicate bacterial growth considered to represent ineffective bacterial killing (i.e. lack of synergy).

### Comparative Example 2: Indifference Between Ceftazidime, Meropenem and Zidovudine

The method and bacterial strains were identical to Comparative Example 1. The effects of the combination were examined with ceftazidime at 4 mg/L as the backbone. The same trend as Comparative Example 1 was observed.

| | | AZT | | | | | | |
|---|---|---|---|---|---|---|---|---|
| | Mg/L | 1 | 0.5 | 0.25 | 0.125 | 0.0625 | 0.03125 | MIC |
| Mero | 16 | 0.0315 | 0.0525 | 0.2645 | 0.3155 | 0.4275 | 0.4515 | 1 |
| | 8 | 0.0085 | 0.1375 | 0.3395 | 0.3935 | 0.3985 | 0.4365 | half |
| | 4 | 0.0165 | 0.0255 | 0.3895 | 0.4075 | 0.3975 | 0.4315 | quarter |
| | 2 | 0.0195 | 0.0795 | 0.3895 | 0.4045 | 0.3845 | 0.3985 | 8th |
| | 1 | 0.0215 | 0.0465 | 0.3995 | 0.4155 | 0.3975 | 0.4165 | 16th |
| | 0.5 | 0.2215 | 0.0845 | 0.3305 | 0.3575 | 0.3885 | 0.3475 | 32nd |
| | MIC | 1 | half | quarter | 8th | 16th | 32nd | |

The results reported in the above table are reported as OD600 values determined as described herein.

The MIC for each drug alone and in combination was also calculated in the same manner as Example 3. The ΣFIC=3 and FIC=2 which was indicative of indifference or antagonism between the three antimicrobial agents.

### Example 3: Synergy Between Ceftazidime, Zidovudine and Doxycycline

The method and bacterial strains were identical to Comparative Examples 1 and 2. The effects of the combination of the present invention were examined with ceftazidime at 4 mg/L as the backbone. The following concentrations (mg/L) of AZT (zidovudine—x axis) and doxycycline (doxy—y axis) were used:

| | | AZT | | | | | | |
|---|---|---|---|---|---|---|---|---|
| | Mg/L | 1 | 0.5 | 0.25 | 0.125 | 0.0625 | 0.03125 | MIC |
| Doxy | 1 | −0.00567 | −0.00567 | −0.00667 | −0.00267 | 0.008333 | −0.00467 | 1 |
| | 0.5 | −0.00567 | −0.00167 | 0.056333 | 0.081333 | 0.137333 | 0.113333 | half |
| | 0.25 | −0.00267 | 0.059333 | 0.098333 | 0.165333 | 0.116333 | 0.128333 | quarter |
| | 0.125 | 0.018333 | 0.065333 | 0.123333 | 0.167333 | 0.174333 | 0.191333 | 8th |
| | 0.0625 | 0.105333 | 0.064333 | 0.144333 | 0.219333 | 0.269333 | 0.286333 | 16th |
| | 0.03125 | 0.012333 | 0.037333 | 0.282333 | 0.360333 | 0.379333 | 0.419333 | 32nd |
| | MIC | 1 | half | quarter | 8th | 16th | 32nd | |

The results reported in the above table are reported as 00600 values determined as described herein.

The MIC for each drug alone and in combination was calculated in the same manner as Comparative Example 1. The ΣFIC=0.75 and FIC=0.5. Contrary to Examples 1 and 2 this was indicative of borderline synergy.

| | MIC | | | |
|---|---|---|---|---|
| | Monotherapy | MIC combination | | |
| | (X) | therapy (Y) | Ratio Y:X | |
| Ceftazidime | 32 | 4 | 0.125 | |
| AZT | 1 | 0.125 | 0.125 | |
| Doxycycline | 1 | 0.5 | 0.5 | |
| | | TOTAL | 0.75 | ΣFIC |
| | | (TOTAL/0.5)/[number | 0.5 | FIC |
| | | of drugs] | | |

### Example 4: Synergy Between Ceftazidime, Zidovudine and Rifampicin

The method and bacterial strains were identical to Comparative Examples 1 and 2. The effects of the combination of the present invention were examined with ceftazidime at 4 mg/L as the backbone. The following concentrations (mg/L) of zidovudine (AZT—x axis) and rifampicin (Rif—y axis) were used:

| | | AZT | | | | | | |
|---|---|---|---|---|---|---|---|---|
| | Mg/L | 1 | 0.5 | 0.25 | 0.125 | 0.0625 | 0.03125 | MIC |
| Rif | 4 | 0.0035 | 0.0055 | 0.0035 | 0.0085 | 0.0055 | 0.0045 | 1 |
| | 2 | 0.0005 | 0.0025 | −0.0015 | 0.0005 | 0.0025 | 0.0025 | half |
| | 1 | 0.0005 | 0.0025 | 0.0125 | 0.0225 | 0.1065 | 0.0605 | quarter |
| | 0.5 | 0.0015 | 0.0015 | 0.0585 | 0.1205 | 0.1985 | 0.1975 | 8th |
| | 0.25 | 0.1425 | 0.0135 | 0.1685 | 0.3305 | 0.3785 | 0.3945 | 16th |
| | 0.125 | 0.0665 | 0.0475 | 0.2905 | 0.3645 | 0.3675 | 0.4335 | 32nd |
| | MIC | 1 | half | quarter | 8th | 16th | 32nd | |

The results reported in the above table are reported as OD600 values determined as described herein.

The MIC for each drug alone and in combination was calculated in the same manner as Example 3. The ΣFIC=0.75 and FIC=0.5 which is indicative of borderline synergy.

### Example 5: Synergy Between Ceftazidime, Zidovudine and Colistin

The method and bacterial strains were identical to Comparative Examples 1 and 2. The effects of the combination of the present invention were examined with ceftazidime at 4 mg/L as the backbone. The following concentrations (mg/L) of zidovudine (AZT—x axis) and colistin (CSS—y axis) were used:

| | | AZT | | | | | | |
|---|---|---|---|---|---|---|---|---|
| | Mg/L | 1 | 0.5 | 0.25 | 0.125 | 0.0625 | 0.03125 | MIC |
| CSS | 2 | −0.00617 | −0.00417 | −0.00217 | 0.000833 | −0.00017 | −0.00017 | 1 |
| | 1 | −0.00817 | −0.00117 | −0.00017 | 0.002833 | 0.004833 | 0.000833 | half |
| | 0.5 | 0.088833 | −0.00017 | 0.111833 | 0.148833 | 0.175833 | 0.244833 | quarter |
| | 0.25 | 0.016833 | 0.020833 | 0.353833 | 0.442833 | 0.378833 | 0.413833 | 8th |
| | 0.125 | 0.111833 | 0.046833 | 0.321833 | 0.461833 | 0.434833 | 0.416833 | 16th |
| | 0.0625 | 0.021833 | 0.109833 | 0.371833 | 0.383833 | 0.447833 | 0.431833 | 32nd |
| | MIC | 1 | half | quarter | 8th | 16th | 32nd | |

The results reported in the above table are reported as OD600 values determined as described herein.

The MIC for each drug alone and in combination was calculated in the same manner as Example 3. The ΣFIC=0.65625 and FIC=0.4375 which is indicative of synergy.

Comparative Examples 1 and 2 vs Examples 3 to 5 support the general understanding in the art that synergy is not predictable or foreseeable. Whilst ceftazidime and zidovudine may be a potent combination against (M)DR gram-negative bacteria, exemplified herein by ESBL E. coli, they do not exemplify synergy when combined when any third antimicrobial agent. Synergy is only observed when ceftazidime and zidovudine are combined with doxycycline, levofloxacin, rifampicin and colistin/polymyxin E.

Synergy is especially not predictable at concentrations below MIC as shown for each of Examples 3 to 5 or against (M)DR bacteria (ESBL E. coli); this means that the combinations are an important development in the fight against antimicrobial resistance. Surprisingly they are successful against bacteria that has an enzyme (ESBL) found in strains known not to be killed by many of the antibiotics that those skilled in the art use to treat infections.

### Example 6: Synergy Between Ceftazidime, Doxycycline and Meropenem

The backbone for Example 6 was ceftazidime at 1 mg/L. The isolate for this Example was resistant to cephalosporins and to ceftazidime at 32 mg/L. Hence, the 1 mg/L concentration of ceftazidime represented 1/16^{th} of the MIC for this isolate.

The method and bacterial strains were identical to Comparative Example 1. The effects of the combination of the present invention were examined with ceftazidime at 1 mg/L as the backbone. The following concentrations (mg/L) of doxycycline (x axis) and meropenem (y axis) were tested:

| | | Doxycycline | | | | |
|---|---|---|---|---|---|---|
| | | 0.0625xMIC | 0.125xMIC | 0.25xMIC | 0.5xMIC | 1xMIC |
| | | Meropenem | | | | |
| | | 0.0625 | 0.125 | 0.25 | 0.5 | 1 |
| 0.0625x MIC | 0.00976563 | 0.31375 | 0.38125 | 0.191 | 0.051 | 0.0135 |
| 0.125x MIC | 0.001953125 | 0.3155 | 0.42375 | 0.14925 | 0.0585 | 0.0165 |
| 0.25x MIC | 0.00390625 | 0.2345 | 0.4285 | 0.16 | 0.052 | 0.004 |
| 0.5x MIC | 0.0078125 | 0.14375 | 0.42075 | 0.12875 | 0.02825 | 0.003 |
| 1x MIC | 0.015625 | −0.00075 | 0.08325 | 0.01325 | −0.00225 | −0.00225 |

The results reported in the above table are reported as OD600 values determined as described herein.

The MIC for each drug alone and in combination was calculated in the same manner as Example 3. The ΣFIC=0.438 and FIC=0.2917 which is indicative of synergy.

### Example 7: Synergy Between Ceftazidime, Doxycycline and Fosfomycin

The method was identical to Comparative Example 1. The effects of the combination of the present invention were examined with ceftazidime at 0.5 mg/L as the backbone against K. pneumoniae CPE. The isolate for this Example was resistant to cephalosporins and to ceftazidime at 4 mg/L. Hence, the 0.5 mg/L concentration of ceftazidime represented ⅛^{th} of the MIC for this isolate. The following concentrations (mg/L) of doxycycline (doxy—y axis) and fosfomycin (Fosfo—x axis)) were tested:

| | | Fosfo | | | | |
|---|---|---|---|---|---|---|
| | | 0.0625x | 0.125x | 0.25x | 0.5x | 1x |
| | | MIC | MIC | MIC | MIC | MIC |
| | | Doxy | | | | |
| | | 4 | 8 | 16 | 32 | 64 |
| 0.0625x MIC | 0.03125 | 0.1085 | 0.1315 | 0.001 | 0.024 | 0.00225 |
| 0.125x MIC | 0.0625 | 0.0325 | 0.207 | 0.0105 | 0.00275 | 0.00175 |
| 0.25x MIC | 0.125 | 0.00575 | 0.10875 | 0.0015 | −0.00175 | 0.007 |
| 0.5x MIC | 0.25 | 0.03 | 0.11575 | −0.0005 | 0.003 | 0.0065 |
| 1x MIC | 0.5 | 0.001 | −0.00075 | 0.0005 | 0.003 | 0.00175 |

The results reported in the above table are reported as OD600 values determined as described herein.

The MIC for each drug alone and in combination was calculated in the same manner as Example 3. The ΣFIC=0.630 and FIC=0.42 which is indicative of synergy.

### Example 8: Synergy Between Ceftazidime, Doxycycline and Levofloxacin

The method and bacterial strains were identical to Comparative Example 1 The effects of the combination of the present invention were examined with ceftazidime at 2 mg/L as the backbone, i.e. ⅛^{th} MIC. The following concentrations (mg/L) of doxycycline (x axis) and levofloxacin (y axis) were tested:

| | | Doxycycline | | | | |
|---|---|---|---|---|---|---|
| | | 0.0625x | 0.125x | 0.25x | 0.5x | 1x |
| | | MIC | MIC | MIC | MIC | MIC |
| | | Levofloxacin | | | | |
| | | 0.0625 | 0.125 | 0.25 | 0.5 | 1 |
| 0.0625x MIC | 0.5 | 0.32475 | 0.318 | 0.17075 | 0.06575 | 0.00925 |
| 0.125x MIC | 1 | 0.30725 | 0.30575 | 0.16275 | 0.05925 | 0.01 |
| 0.25x MIC | 2 | 0.32075 | 0.26925 | 0.136 | 0.0645 | 0.01375 |
| 0.5x MIC | 4 | 0.08025 | 0.1585 | 0.12975 | 0.0455 | 0.02 |
| 1x MIC | 8 | 0.007 | 0.009 | 0.0225 | 0.006 | 0.0075 |

The results reported in the above table are reported as OD600 values determined as described herein.

The MIC for each drug alone and in combination was calculated in the same manner as Example 3. The ΣFIC=0.590 and FIC=0.393 which is indicative of synergy.

### Example 9: Synergy Between Ceftazidime, Rifampicin and Fosfomycin

The method was identical to Comparative Example 1. The effects of the combination of the present invention were examined with ceftazidime at 0.5 mg/L as the backbone and against K. pneumoniae CPE. A ceftazidime concentration of 0.5 mg/L represented ⅛^{th} MIC for the isolate tested. The following concentrations (mg/L) of fosfomycin (x axis) and rifampicin (y axis) were tested:

| | | Fosfomycin | | | | |
|---|---|---|---|---|---|---|
| | | 0.0625x | 0.125x | 0.25x | 0.5x | 1x |
| | | MIC | MIC | MIC | MIC | MIC |
| | | Rifampicin | | | | |
| | | 4 | 8 | 16 | 32 | 64 |
| 0.0625x MIC | 0.5 | 0.46125 | 0.64525 | 0.29325 | −0.01275 | 0.2695 |
| 0.125x MIC | 1 | 0.024 | 0.18525 | 0.28 | −0.01475 | −0.01125 |
| 0.25x MIC | 2 | 0.067 | 0.135 | 0.01475 | −0.01225 | −0.01175 |
| 0.5x MIC | 4 | 0.02575 | 0.03 | −0.00325 | −0.012 | −0.01225 |
| 1x MIC | 8 | 0.01275 | 0.00875 | −0.00725 | −0.007 | −0.0075 |

The results reported in the above table are reported as OD600 values determined as described herein.

The MIC for each drug alone and in combination was calculated in the same manner as Example 3. The ΣFIC=0.354 and FIC=0.236 which is indicative of synergy.

### Example 10: Synergy Between Colistin, Doxycycline and Rifampicin

The backbone for Example 10 was colistin at 0.25 mg/L. This represented ⅛^{th} MIC of the isolate tested (the method and bacterial strain was identical to Comparative Example 1). The following concentrations (mg/L) of doxycycline (x axis) and rifampicin (y axis) were tested:

| | | Doxycycline | | | | |
|---|---|---|---|---|---|---|
| | | 0.0625x | 0.125x | 0.25x | 0.5x | 1x |
| | | MIC | MIC | MIC | MIC | MIC |
| | | Rifampicin | | | | |
| | | 0.0625 | 0.125 | 0.25 | 0.5 | 1 |
| 0.0625x MIC | 0.25 | 0.016 | −0.007 | 0.00025 | −0.006 | −0.006 |
| 0.125x MIC | 0.5 | 0.018 | 0.0025 | 0.0045 | −0.00625 | −0.002 |
| 0.25x MIC | 1 | −0.00125 | 0.00075 | 0.0075 | −0.0035 | −0.003 |
| 0.5x MIC | 2 | 0.00275 | −0.00175 | −0.004 | 0.0135 | −0.001 |
| 1x MIC | 4 | 0.00375 | 0.0015 | −0.00175 | −0.0005 | −0.0015 |

The results reported in the above table are reported as OD600 values determined as described herein.

The MIC for each drug alone and in combination was calculated in the same manner as Example 3. The ΣFIC=0.146 and FIC=0.0973 which is indicative of synergy.

### Example 11: Synergy Between Colistin, Fosfomycin and Gentamicin

The backbone for Example 11 was colistin at 0.5 mg/L. The isolate for this Example was K. pneumoniae CPE and was resistant to colistin at ≥8 mg/L. Hence, the 0.5 mg/L concentration of colistin represented 1/16^{th} of the MIC. The method was identical to Comparative Example 1.

The following concentrations (mg/L) of fosfomycin (x axis) and gentamicin (y axis)) were tested:

| | | Fosfomycin | | | | |
|---|---|---|---|---|---|---|
| | | 0.0625x | 0.125x | 0.25x | 0.5x | 1x |
| | | MIC | MIC | MIC | MIC | MIC |
| | | Gentamicin | | | | |
| | | 4 | 8 | 16 | 32 | 64 |
| 0.0625x MIC | 0.125 | −0.00475 | 0.52925 | 0.21375 | −0.00875 | −0.01375 |
| 0.125x MIC | 0.25 | 0.048 | 0.274 | 0.22325 | −0.0125 | −0.012 |
| 0.25x MIC | 0.5 | 0.027 | 0.21475 | 0.02475 | −0.0135 | −0.012 |
| 0.5x MIC | 1 | 0.002 | 0.06 | −0.0125 | −0.01525 | −0.01375 |
| 1x MIC | 2 | 0.00275 | −0.00175 | −0.013 | −0.01025 | −0.009 |

The results reported in the above table are reported as OD600 values determined as described herein.

The MIC for each drug alone and in combination was calculated in the same manner as Example 3. The ΣFIC=0.625 and FIC=0.417 which is indicative of synergy.

### Example 12: Synergy Between Colistin, Fosfomycin and Levofloxacin

The method and bacterial strains were identical to Example 11. The effects of the combination of the present invention were examined with colistin at 0.5 mg/L as the backbone. This represented 1/16^{th} MIC for this isolate. The following concentrations (mg/L) of fosfomycin (Fosfo—y axis) and levofloxacin (x axis) were tested:

| | | Levofloxacin | | | | |
|---|---|---|---|---|---|---|
| | | 0.0625x | 0.125x | 0.25x | 0.5x | 1x |
| | | MIC | MIC | MIC | MIC | MIC |
| | | Fosfo | | | | |
| | | 4 | 8 | 16 | 32 | 64 |
| 0.0625x MIC | 0.015625 | 0.3335 | 0.5005 | 0.31925 | −0.003 | 0.0015 |
| 0.125x MIC | 0.03125 | 0.16525 | 0.3525 | 0.286 | 0.00125 | 0.07375 |
| 0.25x MIC | 0.0625 | 0.28725 | 0.626 | 0.3235 | 0.00525 | 0.00825 |
| 0.5x MIC | 0.125 | 0.17075 | 0.3665 | 0.08225 | 0.0075 | 0.00225 |
| 1x MIC | 0.25 | 0.0045 | 0.14375 | 0.00675 | 0.0035 | 0.001 |

The results reported in the above table are reported as OD600 values determined as described herein.

The MIC for each drug alone and in combination was calculated in the same manner as Example 3. The ΣFIC=0.615 and FIC=0.417 which is indicative of synergy.

### Example 13: Synergy Between Zidovudine, Colistin, Meropenem and Ceftazidime

The method was identical to Comparative Example 1, except the isolate was P. aeruginosa. The isolate was resistant to ceftazidime up to at least 512 mg/L, to colistin up to at least 512 mg/L, to fosfomycin up to and including 64 mg/L, to meropenem up to and including 64 mg/L and resistant to AZT up to at least 512 mg/L. The effects of the combination of the present invention were examined with colistin at 2 mg/L and ceftazidime at 1 mg/L as the backbone. This represented 1/256^{th} MIC for colistin and 1/512^{th} MIC for ceftazidime for this isolate. The following concentrations (mg/L) of meropenem and zidovudine (AZT) were tested:

| | | Meropenem | | | | | | | | |
|---|---|---|---|---|---|---|---|---|---|---|
| | | 4 | 2 | 1 | 0.5 | 0.25 | 0.125 | 0.0625 | 0.03125 | 0.015625 |
| AZT | 64 | −0.52469 | −0.55383 | 5.013664 | 58.7065 | 73.5726 | 71.94024 | 72.98962 | 80.21862 | 79.37329 |
| | 32 | −0.49554 | −0.55383 | 3.556203 | 59.95992 | 80.65586 | 75.4673 | 78.09073 | 80.59756 | 87.03953 |
| | 16 | −0.34979 | −0.40809 | 21.36637 | 60.80525 | 76.05028 | 70.51193 | 76.51667 | 82.40481 | 93.10257 |
| | 8 | −0.64128 | −0.49554 | 31.07306 | 62.84569 | 81.85097 | 76.66242 | 74.94261 | 86.4274 | 89.40062 |
| | 4 | −0.58298 | −0.64128 | 6.733467 | 64.71124 | 85.69867 | 75.26325 | 76.83731 | 89.86701 | 93.21917 |
| | 2 | 0.641283 | −0.34979 | 31.2771 | 66.37275 | 85.72782 | 81.85097 | 84.50355 | 88.52614 | 95.63855 |
| | 1 mg/L | −0.46639 | −0.55383 | 10.93095 | 75.64219 | 88.40955 | 80.42266 | 84.62015 | 91.9949 | 96.54218 |

The results reported in the above table are reported as percentage bacterial kill values based on 00600 values relative to a positive control.

| | MIC | | | |
|---|---|---|---|---|
| | Monotherapy | MIC combination | | |
| | (X) | therapy (Y) | Ratio Y:X | |
| Zidovudine | 512 | 1 | 0.001953 | |
| Meropenem | 32 | 2 | 0.0625 | |
| Colistin | 512 | 2 | 0.003906 | |
| Ceftazidime | 512 | 1 | 0.001953 | |
| | | TOTAL | 0.070313 | ΣFIC |
| | | (TOTAL/0.5)/[number | 0.035157 | FIC |
| | | of drugs] | | |

The MIC for each drug alone and in combination was calculated as shown above. The ΣFIC=0.0703 and FIC=0.0352 which is indicative of synergy.

### Example 14: Synergy Between Zidovudine, Colistin, Meropenem and Fosfomycin

The method was identical to Comparative Example 1. The isolate was P. aeruginosa and the same as Example 13. The effects of the combination of the present invention were examined with zidovudine at 4 mg/L and meropenem at 4 mg/L as the backbone. This represented 1/128^{th} MIC for zidovudine and ⅛^{th} MIC for meropenem for this isolate. The following concentrations (mg/L) of fosfomycin (y axis) and colistin (x axis) were tested:

| | | Colistin | | | | | |
|---|---|---|---|---|---|---|---|
| | | 64 | 32 | 16 | 8 | 4 | 2 |
| Fosfomycin | 128 | 0.072827 | 0.291306 | 0.619026 | 11.67956 | 21.67501 | 48.60264 |
| | 64 | 0.619026 | 0.728266 | 26.0173 | 24.84297 | 26.8366 | 48.19299 |
| | 32 | 0.072827 | 0.291306 | 26.37233 | 26.2904 | 28.69367 | 62.96768 |
| | 16 | 0.482476 | 1.684115 | 38.9076 | 63.78698 | 68.78471 | 79.35366 |
| | 8 | −0.03641 | −0.0091 | 32.18935 | 44.56076 | 57.06873 | 86.59081 |
| | 4 mg/L | 0.154756 | 0.482476 | 85.41648 | 97.37824 | 95.46655 | 100.1092 |

The results reported in the above table are reported as percentage bacterial kill values based on OD600 values relative to a positive control.

| | MIC | | | |
|---|---|---|---|---|
| | Monotherapy | MIC combination | | |
| | (X) | therapy (Y) | Ratio Y:X | |
| Zidovudine | 512 | 1 | 0.001953 | |
| Meropenem | 32 | 4 | 0.125 | |
| Colistin | 512 | 32 | 0.0625 | |
| Fosfomycin | 256 | 4 | 0.015625 | |
| | | TOTAL | 0.205078 | ΣFIC |
| | | (TOTAL/0.5)/[number | 0.102539 | FIC |
| | | of drugs] | | |

The MIC for each drug alone and in combination was calculated as shown above. The ΣFIC=0.205 and FIC=0.103 which is indicative of synergy.

### Example 15: Synergy Between Zidovudine (AZT), Colistin (CSS), and Fosfomycin (Fosfo)

The method was identical to Comparative Example 1. The isolate was A. baumannii. The isolate was resistant to ceftazidime up to at least 512 mg/L, to colistin up to and including 64 mg/L, to fosfomycin up to and including 128 mg/L, to meropenem up to and including 128 mg/L, and resistant to zidovudine up to at least 512 mg/L. The effects of the combination of the present invention were examined with fosfomycin at 32 mg/L as the backbone. This represented ⅛^{th} MIC for this isolate. The following concentrations (mg/L) of zidovudine (x axis), and colistin (y axis) were tested:

| | | Zidovudine | | | | | |
|---|---|---|---|---|---|---|---|
| | | 1 | 0.5 | 0.25 | 0.125 | 0.0625 | 0.03125 |
| CSS | 2 | 5.783354 | 9.638923 | 48.56181 | 57.92534 | 78.48837 | 82.89474 |
| | 1 | 1.560588 | 25.2448 | 62.3317 | 72.24602 | 73.16401 | 80.14076 |
| | 0.5 | 3.029376 | 4.681763 | 71.51163 | 74.8164 | 72.98042 | 79.22277 |
| | 0.25 | 3.580171 | 14.59608 | 71.51163 | 74.26561 | 70.59364 | 73.16401 |
| | 0.125 | 3.947368 | 8.537332 | 73.34761 | 76.28519 | 72.98042 | 76.46879 |
| | 0.06 mg/L | 40.66707 | 15.51408 | 60.67931 | 65.63647 | 71.32803 | 63.80049 |

The results reported in the above table are reported as percentage bacterial kill values based on OD600 values relative to a positive control.

| | MIC | | | |
|---|---|---|---|---|
| | Monotherapy | MIC combination | | |
| | (X) | therapy (Y) | Ratio Y:X | |
| AZT | 512 | 0.5 | 0.000977 | |
| Fosfo | 256 | 32 | 0.125 | |
| CSS | 128 | 0.5 | 0.003906 | |
| | | TOTAL | 0.129883 | ΣFIC |
| | | (TOTAL/0.5)/[number | 0.086589 | FIC |
| | | of drugs] | | |

The MIC for each drug alone and in combination was calculated as shown above. The ΣFFC=0.130 and FIC=0.0866 which is indicative of synergy

The combination of zidovudine, colistin, and fosfomycin was also tested with fosfomycin at 16 mg/L as the backbone against an P. aeruginosa isolate. The isolate was the same as Example 13. The fosfomycin concentration represented ⅛^{th} MIC for this isolate. The following concentrations (mg/L) of zidovudine (x axis), and colistin (y axis) were tested:

| | | Zidovudine | | | | | |
|---|---|---|---|---|---|---|---|
| | | 1 | 0.5 | 0.25 | 0.125 | 0.0625 | 0.03125 |
| CSS | 1 | 15.77857 | 8.851391 | 7.785672 | −0.20722 | −0.38484 | −0.38484 |
| | 0.5 | 9.206631 | 6.897573 | 64.62404 | 64.2688 | 57.34162 | 58.22972 |
| | 0.25 | 22.70574 | 22.70574 | 60.00592 | 73.14979 | 81.49793 | 69.95263 |
| | 0.125 | 8.851391 | 22.3505 | 65.1569 | 72.26169 | 68.17644 | 74.21551 |
| | 0.0625 | 18.44287 | 4.055654 | 71.37359 | 74.57075 | 75.99171 | 72.97217 |
| | 0.03125 mg/L | 14.89047 | 3.167555 | 68.17644 | 71.55121 | 76.16933 | 65.68976 |

The results reported in the above table are reported as percentage bacterial kill values based on OD600 values relative to a positive control.

| | MIC | | | |
|---|---|---|---|---|
| | Monotherapy | MIC combination | | |
| | (X) | therapy (Y) | Ratio Y:X | |
| AZT | 512 | 0.5 | 0.000977 | |
| Fosfo | 128 | 16 | 0.125 | |
| CSS | 512 | 0.5 | 0.000977 | |
| | | TOTAL | 0.126953 | ΣFIC |
| | | (TOTAL/0.5)/[number | 0.084635 | FIC |
| | | of drugs] | | |

The MIC for each drug alone and in combination was calculated as shown above. The ΣFIC=0.127 and FIC=0.846 which is indicative of synergy.

### Example 16: Synergy Between Zidovudine (AZT), Ceftazidime (Ceft), and Meropenem (Mero)

The method was identical to Comparative Example 1. The isolate was P. aeruginosa, the same as Example 13. The effects of the combination of the present invention were examined with meropenem at 4 mg/L as the backbone. This represented ⅛^{th} MIC for this isolate. The following concentrations (mg/L) of ceftazidime (x axis), and zidovudine (y axis) were tested:

| | | Ceftazidime | | | | | | | | |
|---|---|---|---|---|---|---|---|---|---|---|
| | | 4 | 2 | 1 | 0.5 | 0.25 | 0.125 | 0.0625 | 0.03125 | 0.015625 |
| AZT | 128 | −1.01282 | 8.846631 | −1.07482 | −1.03348 | −0.99215 | 9.59074 | 6.593634 | −0.95081 | −0.97148 |
| | 64 | −0.4754 | 7.048367 | −0.45473 | −0.37205 | −0.45473 | −0.53741 | −0.4754 | −0.4754 | −0.43406 |
| | 32 | −0.49607 | −0.49607 | 11.5957 | −0.41339 | −0.43406 | 0.268706 | −0.45473 | −0.49607 | −0.43406 |
| | 16 | −0.45473 | −0.4754 | −0.45473 | −0.18603 | 11.63704 | −0.16536 | −0.39272 | −0.37205 | 0.537412 |
| | 8 | −0.45473 | −0.22737 | 1.157503 | 3.327821 | 3.327821 | −0.16536 | 0.826788 | 0.764779 | 0.268706 |
| | 4 | −0.28938 | −0.39272 | 2.521703 | 3.741215 | 17.17652 | 3.844564 | 10.85159 | 6.779661 | 10.81025 |
| | 2 mg/L | −0.49607 | −0.41339 | 5.436131 | 10.58289 | 19.65688 | 11.09963 | 17.34188 | 8.019843 | 22.63332 |

The results reported in the above table are reported as percentage bacterial kill values based on OD600 values relative to a positive control.

| | MIC | | | |
|---|---|---|---|---|
| | Monotherapy | MIC combination | | |
| | (X) | therapy (Y) | Ratio Y:X | |
| AZT | 512 | 8 | 0.015625 | |
| Ceft | 512 | 0.5 | 0.000977 | |
| Mero | 32 | 4 | 0.125 | |
| | | TOTAL | 0.141602 | ΣFIC |
| | | (TOTAL/0.5)/[number | 0.094401 | FIC |
| | | of drugs] | | |

The MIC for each drug alone and in combination was calculated as shown above. The ΣFIC=0.142 and FIC=0.0944 which is indicative of synergy.

### Example 17: Synergy Between Zidovudine (AZT), Colistin (CSS), and Ceftazidime (Ceft)

The method was identical to Comparative Example 1. The isolate was A. baumanii, the same as Example 15. The effects of the combination of the present invention were examined with ceftazidime at 32 mg/L as the backbone. This represented 1/16^{th} MIC for this isolate. The following concentrations (mg/L) of colistin (x axis), and zidovudine (y axis) were tested:

| | | Colistin | | | | | | | | |
|---|---|---|---|---|---|---|---|---|---|---|
| | | 256 | 128 | 64 | 32 | 16 | 8 | 4 | 2 | 1 |
| AZT | 128 | −0.88485 | −0.95659 | −0.43047 | −0.83702 | 1.028339 | −0.38264 | 27.16728 | 76.7428 | 85.2804 |
| | 64 | −0.45438 | −0.52613 | −0.6457 | −0.55004 | −0.52613 | 17.2187 | 44.36207 | 70.50102 | 75.85795 |
| | 32 | −0.50221 | −0.52613 | −0.62179 | −0.52613 | 0.263063 | 6.552672 | 43.85986 | 69.32919 | 77.74722 |
| | 16 | −0.50221 | −0.62179 | −0.62179 | −0.33481 | −0.19132 | 4.806887 | 33.81562 | 68.01387 | 77.93854 |
| | 8 | −0.57396 | −0.66962 | −0.71745 | −0.69353 | −0.4783 | 7.126629 | 37.59416 | 68.85089 | 79.87564 |
| | 4 | −0.43047 | −0.1674 | −0.43047 | −0.52613 | 1.506636 | 13.48798 | 31.04149 | 64.40273 | 78.10594 |
| | 2 mg/L | −0.55004 | −0.62179 | −0.62179 | 0.143489 | 0.119574 | 3.682889 | 35.20268 | 69.06612 | 76.52756 |

The results reported in the above table are reported as percentage bacterial kill values based on OD600 values relative to a positive control.

| | MIC | | | |
|---|---|---|---|---|
| | Monotherapy | MIC combination | | |
| | (X) | therapy (Y) | Ratio Y:X | |
| AZT | 512 | 4 | 0.007813 | |
| CSS | 128 | 32 | 0.25 | |
| Ceft | 512 | 32 | 0.0625 | |
| | | TOTAL | 0.320313 | ΣFIC |
| | | (TOTAL/0.5)/[number | 0.213542 | FIC |
| | | of drugs] | | |

The MIC for each drug alone and in combination was calculated as shown above. The ΣFIC=0.320 and FIC=0.214 which is indicative of synergy.

The combination of zidovudine, colistin, and ceftazidime was also tested with colistin at 4 mg/L as the backbone against P. aeruginosa. This represented 1/128^{th} MIC for this isolate. The isolate was the same as Example 13. The following concentrations (mg/L) of ceftazidime (x axis), and zidovudine (y axis) were tested:

| | | Ceftazidime | | | | | | | | |
|---|---|---|---|---|---|---|---|---|---|---|
| | | 256 | 128 | 64 | 32 | 16 | 8 | 4 | 2 | 1 |
| AZT | 128 | −0.54056 | −0.54056 | −0.5856 | −0.49551 | −0.09009 | −0.5856 | −0.56308 | 0.067569 | −0.40542 |
| | 64 | −0.45046 | −0.49551 | −0.54056 | −0.54056 | −0.49551 | −0.49551 | −0.54056 | −0.47299 | 1.328866 |
| | 32 | −0.47299 | −0.56308 | −0.5856 | −0.51803 | −0.51803 | −0.56308 | −0.56308 | −0.27028 | −0.40542 |
| | 16 | 1.306343 | 1.936991 | 14.95538 | 1.914468 | 1.554098 | 0.968496 | 0.495509 | 0.405417 | −0.20271 |
| | 8 | −1.216250 | −1.98204 | −2.00456 | −1.95951 | −1.82438 | −1.64419 | −1.46401 | −0.90093 | 0.292801 |
| | 4 | 0.42794 | 0.653172 | 0.653172 | 0.518033 | 0.247755 | 10.18047 | −0.54056 | 0.450463 | 2.387455 |
| | 2 mg/L | 0.67569 | −0.56308 | −0.49551 | −0.56308 | −0.45046 | −0.51803 | −0.54056 | 0.42794 | 0.247755 |

The results reported in the above table are reported as percentage bacterial kill values based on OD600 values relative to a positive control.

| | MIC | | | |
|---|---|---|---|---|
| | Monotherapy | MIC combination | | |
| | (X) | therapy (Y) | Ratio Y:X | |
| AZT | 512 | 2 | 0.003906 | |
| Ceft | 512 | 1 | 0.001953 | |
| CSS | 512 | 4 | 0.007813 | |
| | | TOTAL | 0.013672 | ΣFIC |
| | | (TOTAL/0.5)/[number | 0.009115 | FIC |
| | | of drugs] | | |

The MIC for each drug alone and in combination was calculated as shown above. The ΣFIC=0.0137 and FIC=0.00912 which is indicative of synergy.

### Example 18: Synergy Between Ceftazidime (Ceft), Levofloxacin (Levo), and Fosfomycin (Fosfo)

The method was identical to Comparative Example 1. The isolate was Methicillin-resistant Staphylococcus aureus (MRSA). The effects of the combination of the present invention were examined with fosfomycin at 2 mg/L as the backbone. This represented ⅛^{th} MIC for this isolate. The following concentrations (mg/L) of ceftazidime (x axis), and levofloxacin (y axis) were tested:

| | | Ceftazidime | | | | | |
|---|---|---|---|---|---|---|---|
| | | 32 | 16 | 8 | 4 | 2 | 1 |
| Levo | 1 | 0.122963 | 0.153704 | 0.030741 | 0.245927 | 0.184445 | 0.922226 |
| | 0.5 | 0.399631 | 0.215186 | 0.245927 | 0.338149 | 0.215186 | 0.737781 |
| | 0.25 | 1.07593 | 0.645558 | 9.560406 | 17.3071 | 15.98524 | 12.32708 |
| | 0.125 | 0.36889 | 1.168152 | 14.14079 | 11.65078 | 13.71042 | 14.50968 |
| | 0.0625 | 1.229634 | 22.10267 | 23.48601 | 31.23271 | 35.19828 | 46.26499 |
| | 0.03125 mg/L | 26.99047 | 37.10421 | 65.20135 | 74.9462 | 82.96957 | 94.98924 |

The results reported in the above table are reported as percentage bacterial kill values based on OD600 values relative to a positive control.

| | MIC | | | |
|---|---|---|---|---|
| | Monotherapy | MIC combination | | |
| | (X) | therapy (Y) | Ratio Y:X | |
| Ceft | 32 | 16 | 0.5 | |
| Levo | 1 | 0.125 | 0.125 | |
| fosfo | 16 | 2 | 0.125 | |
| | | TOTAL | 0.75 | ΣFIC |
| | | (TOTAL/0.5)/[number | 0.5 | FIC |
| | | of drugs] | | |

The MIC for each drug alone and in combination was calculated as shown above. The ΣFIC=0.75 and FIC=0.5 which is indicative of synergy.

### Example 19: Synergy Between Ceftazidime (Ceft), Levofloxacin (Levo), and Meropenem (Mero)

The method was identical to Comparative Example 1. The isolate was MRSA (the same as Example 18). The effects of the combination of the present invention were examined with meropenem at 4 mg/L as the backbone. This represented ⅛^{th} MIC for this isolate. The following concentrations (mg/L) of ceftazidime (x axis), and levofloxacin (y axis) were tested:

| | | Ceftazidime | | | | | |
|---|---|---|---|---|---|---|---|
| | | 32 | 16 | 8 | 4 | 2 | 1 |
| Levo | 1 | 1.01903 | 2.529159 | 2.308165 | 4.849601 | 3.044813 | 1.424187 |
| | 0.5 | 2.124002 | 2.713321 | 6.654389 | 3.560467 | 2.565991 | 5.770411 |
| | 0.25 | 7.906691 | 4.260282 | 4.849601 | 7.170043 | 3.15531 | 5.880909 |
| | 0.125 | 3.00798 | 1.203192 | 35.49417 | 47.98036 | 86.65439 | 99.76673 |
| | 0.0625 | 7.28054 | 4.923266 | 80.68754 | 69.52732 | 81.49785 | 103.4868 |
| | 0.03125 mg/L | 4.812769 | 76.19398 | 83.44997 | 102.787 | 93.54205 | 92.8054 |

The results reported in the above table are reported as percentage bacterial kill values based on OD600 values relative to a positive control.

| | MIC | | | |
|---|---|---|---|---|
| | Monotherapy | MIC combination | | |
| | (X) | therapy (Y) | Ratio Y:X | |
| Ceft | 32 | 1 | 0.03125 | |
| Levo | 1 | 0.25 | 0.25 | |
| Mero | 32 | 4 | 0.125 | |
| | | TOTAL | 0.40625 | ΣFIC |
| | | (TOTAL/0.5)/[number | 0.27083 | FIC |
| | | of drugs] | | |

The MIC for each drug alone and in combination was calculated as shown above. The ΣFIC=0.406 and FIC=0.271 which is indicative of synergy.

The Examples support the combinations of the invention being synergistic. Synergy is not an expected result when combining antimicrobial agents and certainly not when combining three or more antimicrobial agents and/or against bacteria which is multi drug resistant such as ESBL and CPE strains, or in the ESKAPE pathogens. The Examples support the combinations of the invention being synergistic against drug-resistant bacteria, including ESKAPE pathogens, and hence providing a solution to the worldwide problem of antimicrobial resistance as discussed hereinabove. This is a significant advance in the art. The various embodiments described herein are presented only to assist in understanding and teaching the claimed features. These embodiments are provided as a representative sample of embodiments only, and are not exhaustive and/or exclusive. It is to be understood that advantages, embodiments, examples, functions, features, structures, and/or other aspects described herein are not to be considered limitations on the scope of the invention as defined by the claims or limitations on equivalents to the claims, and that other embodiments may be utilised and modifications may be made without departing from the scope of the claimed invention. Various embodiments of the invention may suitably comprise, consist of, or consist essentially of, appropriate combinations of the disclosed elements, components, features, parts, steps, means, etc., other than those specifically described herein. In addition, this disclosure may include other inventions not presently claimed, but which may be claimed in future.

1. An antimicrobial combination comprising three antimicrobial agents, wherein:
i) the first antimicrobial agent is selected from ceftazidime, polymyxin E, polymyxin B, and pharmaceutically acceptable derivatives thereof;
ii) the second antimicrobial agent is selected from zidovudine, doxycycline, fosfomycin and pharmaceutically acceptable derivatives thereof; and
iii) the third antimicrobial agent is selected from levofloxacin, doxycycline, fosfomycin, meropenem, rifampicin, gentamicin, polymyxin B, polymyxin E, and pharmaceutically acceptable derivatives thereof;
wherein the first, second and third antimicrobial agents in the combination are different from one another; wherein the combination includes at least one of levofloxacin, doxycycline, rifampicin, fosfomycin, or a pharmaceutically acceptable derivative thereof; provided the combination is not (1) (i) polymyxin E/B, (ii) zidovudine and (iii) rifampicin or (2) (i) ceftazidime, (ii) zidovudine and (iii) fosfomycin.

2. The combination according to claim 1, wherein the first antimicrobial agent is ceftazidime or a pharmaceutically acceptable derivative thereof.

3. The combination according to claim 1, wherein the second antimicrobial agent is zidovudine or a pharmaceutically acceptable derivative thereof.

4. The combination according to claim 1, wherein the third antimicrobial agent is selected from levofloxacin, doxycycline, rifampicin, polymyxin E, and pharmaceutically acceptable derivatives thereof, preferably wherein the third antimicrobial agent is selected from doxycycline, rifampicin, polymyxin E, and pharmaceutically acceptable derivatives thereof.

5. The combination according to claim 1, wherein the second antimicrobial agent is fosfomycin or a pharmaceutically acceptable derivative thereof.

6. The combination according to claim 1, wherein the third antimicrobial agent is rifampicin, doxycycline or a pharmaceutically acceptable derivative thereof.

7. The combination according to claim 1, wherein the second antimicrobial agent is doxycycline or a pharmaceutically acceptable derivative thereof.

8. The combination according to claim 1, wherein the third antimicrobial agent is meropenem, levofloxacin, or a pharmaceutically acceptable derivative thereof.

9. The combination according to claim 1, wherein the first antimicrobial agent is polymyxin E or a pharmaceutically acceptable derivative thereof, the second antimicrobial agent is doxycycline or a pharmaceutically acceptable derivative thereof, and the third antimicrobial agent is rifampicin or a pharmaceutically acceptable derivative thereof, or wherein the first antimicrobial agent is polymyxin E/B or a pharmaceutically acceptable derivative thereof, the second antimicrobial agent is zidovudine or a pharmaceutically acceptable derivative thereof, and the third antimicrobial agent is fosfomycin or a pharmaceutically acceptable derivative thereof.

10. An antimicrobial combination comprising three antimicrobial agents, wherein the first antimicrobial agent is ceftazidime or a pharmaceutically acceptable derivative thereof; the second antimicrobial agent is zidovudine or a pharmaceutically acceptable derivative thereof; and the third antimicrobial agent is polymyxin E or a pharmaceutically acceptable derivative thereof.

11. The combination according to claim 1, wherein the combination includes a fourth antimicrobial agent which is a carbapenem or a pharmaceutically acceptable derivative thereof, preferably meropenem or a pharmaceutically acceptable derivative thereof.

12. The combination according to claim 11, wherein the first antimicrobial agent is polymyxin E/B or a pharmaceutically acceptable derivative thereof, the second antimicrobial agent is zidovudine or a pharmaceutically acceptable derivative thereof, the third antimicrobial agent is fosfomycin or a pharmaceutically acceptable derivative thereof, and the fourth antimicrobial agent is meropenem or a pharmaceutically acceptable derivative thereof.

13. The combination according to claim 11, wherein the first antimicrobial agent is ceftazidime or a pharmaceutically acceptable derivative thereof; the second antimicrobial agent is zidovudine or a pharmaceutically acceptable derivative thereof; the third antimicrobial agent is polymyxin E/B or a pharmaceutically acceptable derivative thereof, and the fourth antimicrobial agent is meropenem or a pharmaceutically acceptable derivative thereof.

14. An antimicrobial combination comprising three antimicrobial agents, wherein the first antimicrobial agent is ceftazidime or a pharmaceutically acceptable derivative thereof; the second antimicrobial agent is zidovudine, levofloxacin, or a pharmaceutically acceptable derivative thereof; and the third antimicrobial agent is meropenem or a pharmaceutically acceptable derivative thereof.

15. The combination according to claim 1, wherein the pharmaceutically acceptable derivative of polymyxin E is colistin sulfate, colistin methanesulfonate, or colistin methane sulfonate sodium.

16. (canceled)

17. A pharmaceutical composition comprising the combination according to claim 1 and a pharmaceutically acceptable adjuvant, diluent or carrier, preferably for use in the treatment of an infection caused by gram-negative or gram-positive bacteria.

18-21. (canceled)

22. A product comprising the antimicrobial combination according to claim 1, as a combined preparation for simultaneous, separate or sequential use in treating an infection caused by gram-negative or gram-positive bacteria.

23. A product comprising the antimicrobial combination according to claim 10, as a combined preparation for simultaneous, separate or sequential use in treating an infection caused by gram-negative or gram-positive bacteria.

24. The product according to claim 22, wherein the combination includes a fourth antimicrobial agent which is a carbapenem or a pharmaceutically acceptable derivative thereof, preferably meropenem or a pharmaceutically acceptable derivative thereof.

25. A product comprising the antimicrobial combination according to claim 14, as a combined preparation for simultaneous, separate or sequential use in treating an infection caused by gram-negative or gram-positive bacteria.
